(19) United States
(12) Patent Application Publication
(10) Pub. No.: US 2013/0237685 A1
(43) Pub. Date: Sep. 12, 2013

(54) LINEAR ASSEMBLIES, BRANCHED ASSEMBLIES, MACROCYCLES AND COVALENT BUNDLES OF FUNCTIONALIZED BIS-PEPTIDES

(71) Applicants: Christian E. Schafmeister, Merion Station, PA (US); Matthew F. Parker, Zelienopole, PA (US)

(72) Inventors: Christian E. Schafmeister, Merion Station, PA (US); Matthew F. Parker, Zelienopole, PA (US)

(73) Assignees: University Of Pittsburgh - Of The Commonwealth System Of Higher Education, Pittsburgh, PA (US); Temple University - Of The Commonwealth System Of Higher Education, Philadelphia, PA (US)

(21) Appl. No.: 13/885,851

(22) PCT Filed: Nov. 16, 2011
(86) PCT No.: PCT/US11/61012

§ 371 Date: May 16, 2013

Related U.S. Application Data
(60) Provisional application No. 61/414,201, filed on Nov. 16, 2010.

(51) Int. Cl. C07K 7/02; C07K 7/56; C07K 14/00

(52) U.S. Cl. CPC C07K 7/02; C07K 14/001; C07K 7/56
USPC 530/317; 530/323

(57) ABSTRACT

Provided is a macromolecule comprising two or more functionalized bis-peptides connected by one or more linkers, and methods of making thereof. In some embodiments, the functionalized bis-peptides are covalently attached to one or more functionalized bis-peptides to form linear strings of functionalized bis-peptides, macrocycles of functionalized bis-peptides, three-dimensional networks of functionalized bis-peptides, and combinations of any of these. Also provided is a macromolecule comprising two or more bis-peptides connected by one or more linkers, and methods of making thereof. In some embodiments, the bis-peptides comprise non-functionalized bis-peptides, functionalized bis-peptides or a combination of both functionalized and non-functionalized bis-peptides. In some embodiments, the bis-peptides are covalently attached to one or more bis-peptides to form linear strings of bis-peptides, macrocycles of bis-peptides, three-dimensional networks of bis-peptides, and combinations of any of these.

### CROSS-REFERENCE TO RELATED APPLICATIONS

This application claims the benefit of copending U.S. Provisional Application, No. 61/414,201, filed Nov. 16, 2010, the entire disclosure of which is incorporated herein by reference.

### GOVERNMENT SUPPORT

This invention was made with government support under Grant No. HDTRA-09-1-0009 awarded by the Department of Defense. The government has certain rights in this invention.

### FIELD

The present invention relates to shape-programmable macromolecular bundles created by linking bis-peptides optionally containing functional groups through one or more flexible linkers. The resulting macromolecules are shape-programmable and function-programmable synthetic analogs of proteins. These molecules will be used to develop enzyme-like catalysts, sensors for small molecules, therapeutics that bind protein surfaces like antibodies do and molecular devices.

### BACKGROUND

Bis-peptides are analogs of peptides, but are derived from stereochemically pure bis-amino acids bearing two carboxyl groups and two amino groups. The stereochemistry of every bis-amino acid is controlled in its synthesis and can be any combination of (S) and (R) stereochemistry. The connection of specific bis-amino acids leads to the formation of bis-peptides with well-defined molecular shapes, which are of great interest for designing nano-structures. Bis-peptides may be spiro-cyclic oligomers or oligomers assembled from stereochemically pure, cyclic bis-amino acids. Such bis-amino acids display two alpha-amino acid groups mounted on a cyclic core. In the assembly of bis-peptides, diketopiperazine rings are formed between adjacent monomers to create spiro-ladder oligomers with well-defined three-dimensional structures. The advantage of bis-peptides is that the relative position of each monomer's functional group is defined by the monomer's ring structure and stereochemistry in relation to its two immediate neighbors. This is in contrast to proteins, DNA, RNA and unnatural foldamers in which each monomer is joined to its neighbors by one bond and they are highly flexible and disordered. If a particular protein, DNA strand, RNA strand or synthetic foldamer molecule adopts a well-defined three-dimensional structure it does so only after a complex, cooperative folding process that is challenging to accurately model and the outcome of which is difficult to predict.

There remains a need to create macromolecules formed by linking functionalized bis-peptides.

### SUMMARY

Provided is a macromolecule comprising two or more functionalized bis-peptides connected by one or more linkers. In some embodiments, the functionalized bis-peptides are covalently attached to one or more functionalized bis-peptides to form linear strings of functionalized bis-peptides, macrocyles of functionalized bis-peptides, three-dimensional networks of functionalized bis-peptides, and combinations of any of these. In further embodiments, the functionalized bis-peptides are connected by the one or more linkers to form a linear array. In some embodiments, the functionalized bis-peptides are connected by the one or more linkers to form a branched array. In further embodiments, the functionalized bis-peptides are connected by the one or more linkers to form a macrocycle. In further embodiments, the functionalized bis-peptides are connected by the one or more linkers to form a three-dimensional network. In some embodiments, the three-dimensional network comprises pre-organized pockets. In further embodiments, the three-dimensional network comprises dendrimers. In yet further embodiments, the functionalized bis-peptides are functionalized with reactive groups comprising amines, carboxylic acids, azides, alkynes, thiols, olefins, dienes, dienophiles, aldehydes, hydrazines, phosphines, maleimides, haloacetates, halobenzyl groups, alcohols, aziridines, epoxides, aryl halides or vinyl halides.

In some embodiments, one or more of the linkers are flexible. In further embodiments, one or more of the linkers are branched. In yet further embodiments, the one or more linkers comprise amides, esters, cis or trans alkenes, amino acids, amines, ethers, thioethers, acyls, allyls, propargyls, benzyls, hydrazines, triazoles, rings using Diels-Alder couplings, hydrazones, hydrazides, disulphide linkages, alkyl-chains, aromatic rings, olefins formed using metathesis, phosphonate ester linkages, silyl linkages, organometallic linkages and alkyl chain linkages and combinations thereof. In some embodiments, the one or more linkers are formed by reaction of reactive groups located on the two or more bis-peptides. In further embodiments, the reactive groups are located on opposite ends of said two or more bis-peptides. In further embodiments, one reactive group is located on an interior portion of the one or more bis-peptides and one reactive group is located on an end of the two or more bis-peptides. In yet further embodiments, the reactive groups are located on an interior portion of the two or more bis-peptides. In some embodiments, the two reactive groups are located on the same end of the two or more bis-peptides.

Provided is a method of making a functional macromolecule comprising assembling functionalized bis-peptides through multiple linkers. In some embodiments, one or more of the linkers are flexible. In some embodiments, one or more of the linkers are branched.

Provided is a method of making a macromolecule comprising at least two functionalized bis-peptides joined to each other through a flexible linker comprising covalently attaching a functionalized bis-peptide to one or more functionalized bis-peptides to form linear strings of functionalized bis-peptides, macrocycles of functionalized bis-peptides, three-dimensional networks of functionalized bis-peptides, and combinations of any of these. In some embodiments, one or more of the linkers are flexible. In some embodiments, one or more of the linkers are branched.

Provided is a method of making a macromolecule comprising two or more functionalized bis-peptides connected by one or more linkers comprising reacting two or more reactive groups on the one or more functionalized bis-peptides to form the macromolecule. In some embodiments, one or more of the linkers are flexible. In some embodiments, one or more of the linkers are branched. In further embodiments, the reactive groups are on opposite ends of the two or more bis-peptides. In yet further embodiments, one reactive group is on an interior portion of the one or more bis-peptides and one reactive group is on an end of the two or more bis-peptides. In some embodiments, two reactive groups are on an interior portion of the two or more bis-peptides. In further embodiments, two reactive groups are on the same end of the two or more bis-peptides.

Provided is a macromolecule comprising two or more bis-peptides connected by one or more linkers. In some embodiments, the bis-peptides comprise non-functionalized bis-peptides, functionalized bis-peptides or a combination of both functionalized and non-functionalized bis-peptides. In some embodiments, the bis-peptides are covalently attached to one or more bis-peptides to form linear strings of bis-peptides, macrocycles of bis-peptides, three-dimensional networks of bis-peptides, and combinations of any of these. In some embodiments, the bis-peptides are connected by the one or more linkers to form a linear array. In further embodiments, the bis-peptides are connected by the one or more linkers to form a branched array. In yet further embodiments, the bis-peptides are connected by the one or more linkers to form a macrocycle. In some embodiments, the bis-peptides are connected by the one or more linkers to form a three-dimensional network.

Provided is a method of making a macromolecule comprising two or more bis-peptides connected by one or more linkers comprising reacting two or more reactive groups on the one or more bis-peptides to form the macromolecule. In some embodiments, the bis-peptides comprise non-functionalized bis-peptides, functionalized bis-peptides or a combination of both functionalized and non-functionalized bis-peptides. In some embodiments, one or more of the linkers are flexible. In further embodiments, one or more of the linkers are branched.

As envisioned in the present invention with respect to the disclosed compositions of matter and methods, in one aspect the embodiments of the invention comprise the components and/or steps disclosed therein. In another aspect, the embodiments of the invention consist essentially of the components and/or steps disclosed therein. In yet another aspect, the embodiments of the invention consist of the components and/or steps disclosed therein.

### BRIEF DESCRIPTION OF THE DRAWINGS

FIG. 1 illustrates the acylation of a hindered amine.

FIG. 2 illustrates the formation of a bis-peptide having a diketopiperazine ring from a hindered amine and an acyl.

FIG. 3 illustrates the preparation of a hexasubstituted diketopiperazine.

FIG. 4 A illustrates bis-amino acid monomers. The curly lines across two bonds represent a tetra-, penta- or hexa-substituted diketopiperazine. R, R₁, R₂, R₃, R₄, R₅ are functional groups or linkers (L). FIG. 4 B illustrates how the ends of bis-peptides can be functionalized to connect to the linker (L).

FIG. 5 A illustrates schematic representations of bis-peptide oligomers and linking groups. FIG. 5 B illustrates linear arrays of bis-peptide bundles connected by linkers. FIG. 5 C illustrates macrocyclic assemblies of bis-peptides. FIG. 5 D illustrates how three-dimensional networks of bis-peptides can be joined. FIG. 5 E illustrates branched assemblies, which are a type of three-dimensional network of bis-peptides.

FIG. 6 illustrates the coupling of three bis-peptides through an amide linkage to a polyamine. Tris(aminoethyl)amine was used to make a three oligomer branched structure.

FIG. 7 A-B illustrates reverse phase C18 HPLC-MS analysis of Compound 33 (Preparative Example 1): Eluted on a gradient of 5% MeCN to 95% MeCN in H₂O over 30 min. m+1=771.3, found: 770.8.

FIG. 8 A-B illustrates reverse phase C18 HPLC-MS analysis of Compound 41 (Preparative Example 2): Eluted on a gradient of 5% MeCN to 95% MeCN in H₂O over 30 min. m+23=952.4, found: 952.0.

FIG. 9 A-B illustrates reverse phase C18 HPLC-MS analysis of Compound 47 (Preparative Example 3): Eluted on a gradient of 5% MeCN to 95% MeCN in H₂O over 30 min. m+1=911.3, found: 911.

FIG. 10 A-B illustrates reverse phase C18 HPLC-MS analysis of Compound 49 (Example 6): Eluted on a gradient of 5% MeCN to 95% MeCN in H₂O over 30 min. m+1=2453.0, found: 2452.6.

FIG. 11 A-B illustrates reverse phase C18 HPLC-MS analysis of Compound 6 (Example 2): Eluted on a gradient of 5% MeCN to 95% MeCN in H₂O over 30 min. m+1=1506.46, found: 1506.2.

FIG. 12 A-B illustrates reverse phase C18 HPLC-MS analysis of Compound 9 (Example 3): Eluted on a gradient of 5% MeCN to 95% MeCN in H₂O over 30 min. m+1=2104.67, found: 2104.5.

FIG. 13 A-B illustrates reverse phase C18 HPLC-MS analysis of Compound 65 (Example 7): Eluted on a gradient of 5% MeCN to 95% MeCN in H₂O over 30 min. m+1=1626.5, found: 1626.9.

FIG. 14 A-B illustrates reverse phase C18 HPLC-MS analysis of Compound 10 (Example 4): Eluted on a gradient of 5% MeCN to 95% MeCN in H₂O over 30 min. m+1=1424.53, found: 1424.3.

FIG. 15 A-B illustrates reverse phase C18 HPLC-MS analysis of Compound 11 (Example 4): Eluted on a gradient of 5% MeCN to 95% MeCN in H₂O over 30 min. m+1=2023.75, found: 2023.7.

FIG. 16 A-B illustrates reverse phase C18 HPLC-MS analysis of Compound 69 (Example 7): Eluted on a gradient of 5% MeCN to 95% MeCN in H₂O over 30 min. m+1=1545.6, found: 1545.9.

FIG. 17 A-B reverse phase C18 HPLC-MS analysis of Compound 14 (Example 5): Eluted on a gradient of 0% MeCN to 50% MeCN in H₂O over 30 min. m+2/2=1085.80, found: 1085.8.

FIG. 18 A-B illustrates reverse phase C18 HPLC-MS analysis of 15 (Example 5): Eluted on a gradient of 0% MeCN to 50% MeCN in H₂O over 30 min. m+2/2=1005.38, found: 1005.2.

### DEFINITIONS

The articles “a” and “an” are used herein to refer to one or to more than one (i.e. to at least one) of the grammatical object of the article. By way of example, “an element” means one element or more than one elements.

The term “about” will be understood by persons of ordinary skill in the art and will vary to some extent depending on the context in which it is used. As used herein, “about” is meant to encompass variations of ±20% or ±10%, more preferably ±5%, even more preferably ±1%, and still more preferably ±0.1%.

### GLOSSARY OF ABBREVIATIONS

AcOH: acetic acid

### Alloc-Cl: Allylchloroformate

At: 7-azabenzotriazole
BH₃:DMA: Borane-Dimethylamine complex
Bn: benzyl
Boc (also referred to as tBoc): t-butyloxycarbonyl
Boc-Gly-OH: 2-(tert-butoxycarbonylamino)acetic acid
Bt: benzotriazole
Cbz: carbobenzyloxy
DCC: dicyclohexylcarbodiimide
DCM: dichloromethane
DEA: diethylamine
DIC: diisopropylcarbodiimide
DIPEA: diisopropylethylamine
Dmab: 4-{N-[1-(4,4-dimethyl-2,6-dioxocyclohexylidene)-3-methylbutyl]amino}benzyl ester
DMAP: dimethylaminopyridine

### DMF: N,N-Dimethylformamide

### EtOAc: Ethylacetate

### Fmoc: 9-Fluorenylmethyloxycarbonyl

Fmoc-Dab(Fmoc)-OH: (S)-2,4-bis(((9H-fluoren-9-yl)methoxy)carbonylamino)butanoic acid
Fmoc-Nal-OH: (S)-2-(9H-fluoren-9-yl)methoxy)carbonylamino)-3-(naphthalen-1-yl)propanoic acid
Fmoc-D-Dab(IvDde)-OH: (R)-2-(9H-fluoren-9-yl)methoxy)carbonylamino)-4-(1-(4,4-dimethyl-2,6-dioxocyclohexylidene)-3-methylbutylamino)butanoic acid
Fmoc-Dab(Boc)-OH: (S)-2-(9H-fluoren-9-yl)methoxy)carbonylamino)-4-(tert-butoxycarbonylamino)butanoic acid
Fmoc-Gly-OH: 2-(((9H-fluoren-9-yl)methoxy)carbonylamino)acetic acid

### H₂O: Water

HATU: 2-(1H-7-Azabenzotriazol-1-yl)-1,1,3,3-tetramethyl uronium hexafluorophosphate Methanaminium
HMBA: 4-hydroxymethylbenzoic acid
HOAT: 1-Hydroxy-7-azabenzotriazole

### IPA: Isopropanol

### MeCN: Acetonitrile

### MeIm: N-Methylimidazole

MSNT: 1-(Mesitylene-2-sulfonyl)-3-nitro-1,2,4-triazole

### NH₄Cl: Ammonium Chloride

### NH₄OAc: Ammonium Acetate

### NaHCO₃: Sodium Bicarbonate

### NaCl: Sodium Chloride

### Na₂SO₄: Sodium Sulfate

### NMP: N-Methylpyrrolidine

Oat/OAt: 1-oxy-7-azabenzotriazole

### (PPh₃)₄Pd: Tetrakis(Triphenylphospine)palladium

Pip: piperidine
PyAOP: (7-Azabenzotriazol-1-yloxy)tripyrrolidinophosphonium hexafluorophosphate

### TEA: Triethylamine

### TFA: Trifluoroacetic Acid

TIS: triisopropylsilane

### TIPS: Triisopropylsilane

### DETAILED DESCRIPTION

Two or more bis-peptides are combined herein through flexible linkers to create multi-bis-peptide macromolecules. In some embodiments exemplified below, the multi-bis-peptides are prepared from non-functionalized bis-peptides. In some embodiments, the multi-bis-peptides are formed from bis-peptides that incorporate functional groups. Such functional groups may be created using the “acyl-transfer coupling” reaction described in WO 2010/009196 A1, which is hereby incorporated by reference in its entirety. In some embodiments, the multi-bis-peptides are formed from a combination of functionalized and non-functionalized bis-peptides.

### Bis-Peptides

In some embodiments, a bis-peptide corresponds to the general structure:

[DKP¹(X¹)a]b-CYCLE¹(Y¹)c-[-DKP²(X²)d-CYCLE²(Y²)e-]f-[-DKP³(X³)g]h

Wherein a is 0 or an integer (e.g. 1-4), b is 0 or 1, c is 0 or 1, d is 0, 1 or 2, e is 0 or 1, f is 0 or an integer (e.g. 1-50), g is 0 or an integer (e.g. 1-4), h is 0 or 1; DKP¹, DKP² and DKP³ are diketopiperazine rings; X¹, X², and X³ are the same or different and are hydrogen or functional groups or linking groups attached to a tertiary amide nitrogen atom or carbon of a diketopiperazine ring, subject to the proviso that when f is greater than 1, X² may differ among the -DKP²(X²)d-CYCLE²(Y²)e- repeating units; and CYCLE¹ and CYCLE² are optional cyclic rings containing carbon, nitrogen, oxygen and hydrogen forming five-membered rings, six-membered rings or fused rings consisting of five and six membered rings the same or different, wherein each CYCLE may be optionally fused to a diketopiperazine ring adjacent to it, subject to the proviso that when f is greater than 1, CYCLE² may differ among the -DKP²(X²)d-CYCLE²(Y²)e- repeating units. Y¹ and Y² are the same or different and are hydrogen or functional groups or linking groups attached to a carbon or nitrogen of a CYCLE, subject to the proviso that when f is greater than 1, Y² may differ among the -DKP²(X²)d—CYCLE²(Y²)e-repeating units. Each X¹ is independently selected from hydrogen, functional groups, rings and linking groups. Each X² is independently selected from hydrogen, functional groups and linking groups. Each X³ is independently selected from hydrogen, functional groups, rings and linking groups. At least two of X¹, Y¹, X², Y² and X³ are flexible linking groups that are linked to each other. It may be appreciated that the bis-peptide may be considered as functionalized when at least one of X¹, X² and X³ is a functional group.

In some embodiments, a bis-peptide corresponds to the general structure shown below, wherein Rx is a protecting group:

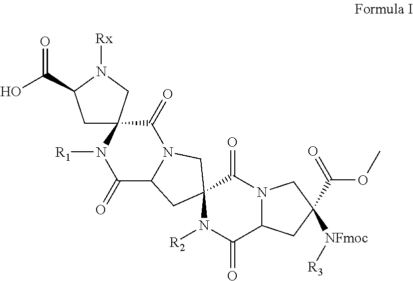

In some embodiments, the bis-peptide according to Formula I is functionalized, wherein at least one of R₁, R₂ and R₃ are other than H and represent functional groups or linkers.

In some embodiments, the bis-peptide according to Formula I is non-functionalized, wherein R₁, R₂ and R₃ are all H. The term “bis-peptide” lacking either the “functionalized” or “non-functionalized” modifier describes a bis-peptide that is selected from functionalized bis-peptides and non-functionalized bis-peptides.

According to the present invention, functionalized bis-peptides bearing functional groups can be covalently connected to other functionalized bis-peptides to create macromolecules (1,000 Daltons and up) with new properties and capabilities. Functionalized bis-peptides can be connected to each other in a linear fashion or through branched linkers to create flexible, multi-domain, linear assemblies of bis-peptides. Functionalized bis-peptides can be connected into macrocyclic rings containing two or more bis-peptides that form triangles, squares and larger shapes. Functionalized bis-peptides can be connected to each other through two or more linkages to form complex networks that form complex three-dimensional structures containing pre-organized pockets and displaying large pre-organized surfaces displaying hundreds and thousands of square angstroms of surface area.

Functionalized bis-peptides may be assembled through multiple flexible linkages to create functional macromolecules. The rigid, pre-organized nature of bis-peptides and the ability to selectively functionalize them with reactive groups including amines, carboxylic acids, azides, alkynes, thiols, olefins, diener, dienophiles, aldehydes, hydrazines, phosphines, maleimides, haloacetates, halobenzyl groups, alcohols, aziridines, epoxides, aryl halides and vinyl halides allows for the combination and cross-linking of bis-peptides into larger structures that approach the complexity of folded natural proteins. Bis-peptides can be cross-linked using different linear and branched linkages including amides, esters, secondary amines, triazoles, rings using Diels-Alder couplings, hydrazones, hydrazides, disulphide linkages, alkyl-chains, aromatic rings, olefins formed using metathesis, phosphonate ester linkages, silyl linkages, organometallic linkages and alkyl chain linkages and combinations of these. Because functionalized bis-peptides may be rigid, and because they can be designed to display functional groups in pre-organized constellations, functionalized bis-peptides can be created that display one or more reactive functional groups in complementary fashion so that they can combine with other functionalized bis-peptides in pre-determined ways to create specific three-dimensional structures.

These covalent bis-peptide bundles can combine protein binding bis-peptide based epitopes to create extended surfaces with high selectivity and binding affinity. Covalent bis-peptide bundles can combine hundreds of bis-peptide based protein binding epitopes to create macromolecules that have long life-times in blood (150,000 Dalton oligomers) and extremely high avidity for their protein targets. Covalent bundles of bis-peptides could organize functional groups around a central pocket to create enzyme-like active sites. Covalent bundles of functionalized bis-peptides could create pre-organized pockets that bind small molecules to create sensors. Materials constructed from cross-linked bis-peptides could be used to filter water and other solvents or gases or separate compounds from each other. These are just a few of the applications that could be developed using cross-linked, functionalized bis-peptides.

In some embodiments, the invention provides a macromolecule comprising at least two functionalized bis-peptides joined to each other through a flexible linker. The component-functionalized bis-peptides may be covalently attached to one or more functionalized bis-peptides to form linear strings of functionalized bis-peptides, branched structures displaying bis-peptides, macrocycles of functionalized bis-peptides, three-dimensional networks of functionalized bis-peptides and combinations of all of these.

### Functionalized Bis-Peptides

Bis-peptides include oligomeric molecules that contain a plurality of diketopiperazine rings, wherein at least one diketopiperazine ring contains a tertiary amide nitrogen atom bearing a pendant functional group. Bis-peptides may be functionalized or non-functionalized, as explained supra. The preparation of some functionalized bis-peptides is described in WO 2010/009196.

The secondary amide nitrogen between every pair of monomers of a bis-peptide is an ideal location for incorporating additional chemical functionality. Utilizing this position, it is possible to incorporate functionality late in the monomer synthesis or even on a solid support during assembly of the bis-peptide. For example, a primary amine group can be alkylated with an alkyl halide or the like or reacted with various aldehydes in a reductive amination to introduce the desired functional group. The oligomer or polymer can then be further extended by reaction with another amino acid. However, this synthetic approach does not work well in practice, as the resulting secondary amine is no longer sufficiently nucleophilic to undergo acylation with the next amino acid.

This problem can be solved by introducing a free carboxylic acid group alpha to the secondary (hindered) amine group in the bis-peptide. The carboxylic acid group apparently facilitates acylation of the secondary amine group under relatively mild conditions, perhaps due to participation by this neighboring group. This result was unexpected, in that a secondary amine group alpha to a carboxylic acid alkyl ester (e.g., —CO₂CH₃) reacts sluggishly, if at all, with amino acids, especially hindered amino acids.

Provided is a unique class of functionalized, dimeric, oligomeric and polymeric compounds (functionalized bis-peptides) built from a collection of building blocks (bis-amino acids) which may be assembled in different sequences and in different lengths. Each building block can display a functional group, although non-functionalized building blocks can also be introduced (as spacer repeating units, for example). The functional groups are pendant to the backbone of the bis-peptide, i.e., they extend out or away from the bis-peptide backbone (sometimes also referred to as the “bis-peptide scaffold”) and thus can be available for interaction with other molecules or chemical species (e.g., complexation, reaction, binding). In one aspect, the functional group is attached to a nitrogen atom. Any sequence of bis-amino acid building blocks may be connected through pairs of amide bonds to create bis-peptides, wherein at least one bis-amino acid building block in the bis-peptide molecule carries a functional group. In one aspect, a plurality of bis-amino acids in the bis-peptide molecule carry functional groups, wherein functional groups of at least two different types are present in the bis-peptide. In another aspect, the functional group is attached to a nitrogen atom that is part of a diketopiperazine ring structure in the bis-peptide. Such functionalized nitrogen atoms thus can have a tertiary amide structure. The functional groups may be introduced using different approaches, including a submonomer approach (where an amine group is functionalized during synthesis of the bis-peptide) as well as an approach where building blocks with the functionality already installed are utilized.

In some embodiments, the bis-peptides are spiroladder macromolecules (oligomers, polymers) having no rotable bonds in their backbones.

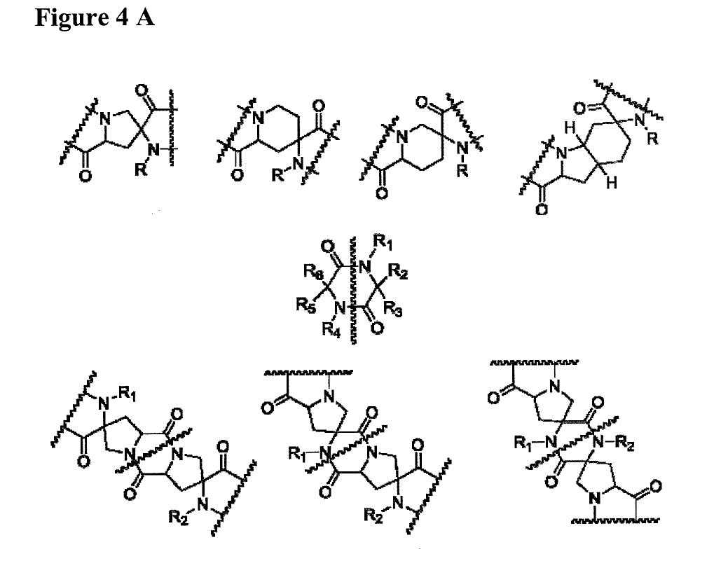

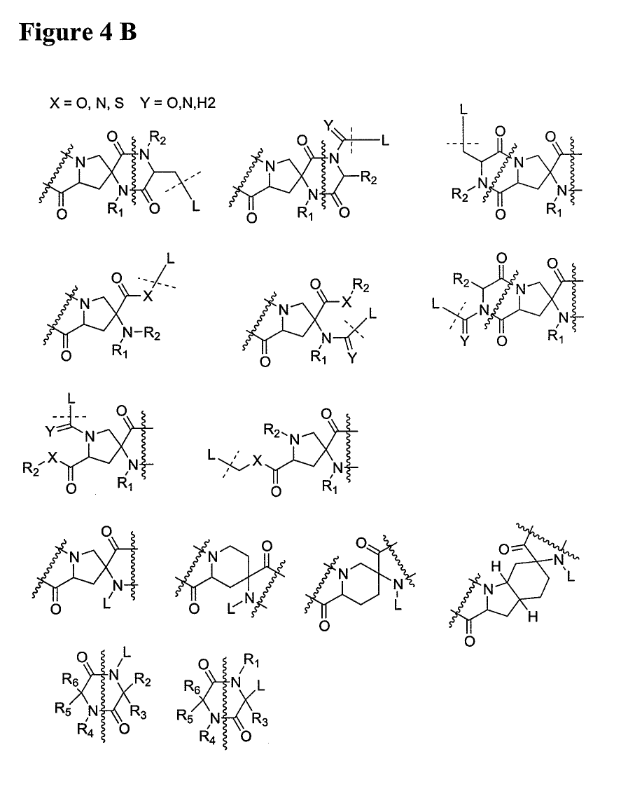

Examples of bis-peptides that may be used to form the macromolecules of the invention are those bis-peptide molecules formed by combining bis-amino acid monomers, such as those shown in FIG. 4 A, through diketopiperazine linkages. These diketopiperazine linkages are easy to form and may be formed by the use of the acyl-transfer coupling reaction described in WO 2010/009196 A1. The curly lines across two bonds in FIG. 4 A represent a tetra-, penta- or hexa-substituted diketopiperazine.

FIG. 4B illustrates certain examples of how the ends of bis-peptides can be functionalized to connect to a linker (L).

### Functional Groups

The functional groups that can be displayed on bis-peptides include, for example, the functional groups described WO 2010/009196 A1. Such functional groups include, for example, aromatic-containing groups (e.g., phenyl, benzyl, p-cresol, 1-methoxy-benzene, naphthyl, imidazole, 4-methyl-phenol, 1-methoxy-4-methyl-benzene, 2-pyrene, 1-methylimidazole, indole, 2-pyridine, 3-pyridine, triazole, imidazole), carboxylic acid-containing groups (e.g., ethanoic acid, acetic acid, propionoic acid), ester-containing groups (e.g., methyl formate, methyl acetate), amide-containing groups (e.g., ethanoamide, propionamide), hydroxamic acid-containing groups (e.g., carboxhydroxamide, ethanohydroxamide, propionhydroxamide), amine-containing groups (e.g., amine, methanamine, ethanamine, propanamine, N,N-dimethylmethanamine, methyl-guanidine, ethyl-guanidine, propyl-guanidine, dimethylamine, N,N,N-trimethylmethanamine, methylamine, methyl-thiourea, ethyl-thiourea, 1-(3,5-bis(trifluoromethyl)phenyl)-3-ethylthiourea, or 1-(3,5-bis(trifluoromethyl)phenyl)-3-ethylurea), azido-containing groups (e.g., methyl-azide, azide), aliphatic-containing groups (e.g., isopropyl, isobutyl, isopentyl, ethyl, methyl, cyclopentyl, cyclohexyl, 1-methyl-propyl), hydroxyl- or sulfuhydryl-containing groups (e.g., hydroxyl, methyl-hydroxyl, thiol, methyl-thiol), ether- or thioether-containing groups (e.g., methyl-ether, ethyl-ether, methyl-thioether, ethyl-thioether), alkenyl or alkynyl groups (e.g., ethene, allyl, ethyne, propargyl), nucleobase-containing groups (e.g., guanine, adenine, cytosine, thymine). In one aspect of the present invention, at least one of the aforementioned functional groups is attached to a carbon atom which in turn is connected to a nitrogen atom, in particular a nitrogen atom that is part of a diketopiperazine moiety contained within the bis-peptide. For example, one of the nitrogen atoms in a diketopiperazine moiety of the bis-peptide may bear a group —CHR¹R², wherein R¹ and R² may be the same or different and may be hydrogen (—H) or one of the aforementioned functional groups. The functional groups may be hydrocarbyl groups (i.e., groups containing only carbon and hydrogen atoms) or substituted hydrocarbyl groups (i.e., groups containing one or more atoms other than carbon and hydrogen atoms, such as oxygen, sulfur, nitrogen and/or halogen atoms). The functional group may be neutral, acidic or basic and may be ionic in character (e.g., a salt).

The preceding list of R¹ and R² groups is exemplary. Additional exemplary functional groups include Ar, (C₁-C₆)-straight or branched alkyl, (C₂-C₆)-straight or branched alkenyl or alkynyl, (C₅-C₇)-cycloalkyl substituted (C₁-C₆)-straight or branched alkyl, (C₅-C₇)-cycloalkyl substituted (C₃-C₆)-straight or branched alkenyl or alkynyl, (C₅-C₇)-cycloalkenyl substituted (C₁-C₆)-straight or branched alkyl, (C₅-C₇)-cycloalkenyl substituted (C₃-C₆)-straight or branched alkenyl or alkynyl, Ar-substituted (C₁-C₆)-straight or branched alkyl, Ar-substituted (C₃-C₆)-straight or branched alkenyl or alkynyl; wherein any one of the CH₂ groups of the alkyl chains is optionally replaced by a heteroatom selected from the group consisting of O, S, SO, SO₂, and NR; wherein R is selected from the group consisting of hydrogen, (C₁-C₄)-straight or branched alkyl, (C₃-C₄)-straight or branched alkenyl or alkynyl, and (C₁-C₄) bridging alkyl wherein a bridge is formed between the nitrogen and a carbon atom of the heteroatom-containing chain to form a ring, and wherein the ring is optionally fused to an Ar group; wherein Ar is a carbocyclic aromatic group selected from the group consisting of phenyl, 1-naphthyl, 2-naphthyl, indenyl, azulenyl, fluorenyl, and anthracenyl; or a heterocyclic aromatic group selected from the group consisting of 2-furyl, 3-furyl, 2-thienyl, 3-thienyl, 2-pyridyl, 3-pyridyl, 4-pyridyl, pyrrolyl, oxazolyl, thiazolyl, imidazolyl, pyraxolyl, 2-pyrazolinyl, pyrazolidinyl, isoxazolyl, isotriazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, benzo[b]thiophenyl, 1H-indazolyl, benzimidazolyl, benzthiazolyl, purinyl, 4H-quinolizinyl, quinolinyl, 1,2,3,4-tetrahydroquinolinyl, isoquinolinyl, 1,2,3,4-tetrahydroisoquinolinyl, cinnolinyl, phthalazinyl, quinazolinyl, quinoxalinyl, 1,8-naphthyridinyl, pteridinyl, carbazolyl, acridinyl, phenazinyl, phenothiazinyl, and phenoxazinyl; wherein Ar is optionally substituted with one or more substituents which are independently selected from the group consisting of hydrogen, halogen, hydroxyl, nitro, —SO₃H, trifluoromethyl, trifluoromethoxy, (C₁-C₆)-straight or branched alkyl, (C₂-C₆)-straight or branched alkenyl, —O—[(C₁-C₆)-straight or branched alkyl], O—[(C₃-C₄)-straight or branched alkenyl], —O-benzyl, —O-phenyl, 1,2-methylenedioxy, —NR⁵R⁶, carboxyl, —N—(C₁-C₅-straight or branched alkyl or C₃-C₅-straight or branched alkenyl) carboxamides, —N,N-di-(C₁-C₅-straight or branched alkyl or C₃-C₅-straight or branched alkenyl) carboxamides, morpholinyl, piperidinyl, —O-M, —CH₂—(CH₂)_{q}-M, —O—(CH₂)_{q}-M, —(CH₂)_{q}—O-M, and —CH═CH-M; wherein R⁵ and R⁶ are independently selected from the group consisting of hydrogen, (C₁-C₆)-straight or branched alkyl, (C₃-C₆)-straight or branched alkenyl or alkynyl and benzyl; wherein M is selected from the group consisting of 4-methoxyphenyl, 2-pyridyl, 3-pyridyl, 4-pyridyl, pyrazyl, quinolyl, 3,5-dimethylisoxazoyl, 2-methylthiazoyl, thiazoyl, 2-thienyl, 3-thienyl and pyrimidyl; and q is 0-2. R¹ and R² may also be linked to each other to form a ring, such as a hydrocarbyl or substituted hydrocarbyl ring (e.g., cyclohexyl, cyclopentyl).

In some embodiments, one or both of the end groups of the bis-peptide may contain a diketopiperazine ring in which a carbon atom in the diketopiperazine ring of the end group bears at least one pendant functional group other than hydrogen. This pendant functional group may be any of the types of functional groups previously mentioned. The stereochemistry of the ring carbon atom to which the functional group or functional groups is or are attached may be selected and controlled as may be desired, e.g., (S) or (R). The functional group at this position may be utilized to introduce a label, such as a fluorescent label (a functional group capable of fluorescing, i.e., a fluorescent tracer such as fluorescein) into the bis-peptide molecule.

The methods described herein allow the creation of highly hindered tertiary amides in peptides and highly substituted diketopiperazines that are very difficult to synthesize by other means. Hindered amide bonds and highly substituted diketopiperazines are valuable as motifs in drug syntesis.

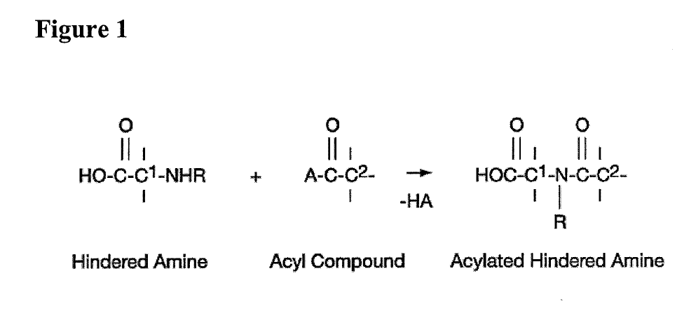

Accordingly, a hindered amide is obtained by acylating a hindered amine, wherein the hindered amine has a secondary amine group and a carboxylic acid group alpha to the secondary amine group and is reacted with an acyl compound containing an activated acyl group. FIG. 1 illustrates this type of reaction, where R is a substituent other than hydrogen such as a hydrocarbyl group, substituted hydrocarbyl group, or protecting group and Z is an activating group such as fluorine, OAt or the like. Substituent R may form part of a ring structure including the nitrogen atom of the secondary amine group and the carbon atom to which the free carboxylic acid group is attached (C¹ in FIG. 1). In one embodiment, the hindered amine bears at least one substituent other than hydrogen and the carboxylic acid group on the carbon atom to which both the secondary amine group and the carboxylic acid group are attached (C¹ in FIG. 1). Such substituents can be any of the functional groups previously described. In some embodiments, the acyl compound is also hindered. For example, the carbon atom adjacent to the C═O group in the acyl compound (C² in FIG. 1) can be substituted with two or more functional groups other than hydrogen, with any of the functional groups previously described being suitable for such purpose (e.g., hydrocarbyl groups and/or substituted hydrocarbyl groups). In one aspect, the acyl compound bears an amine group and at least one substituent other than hydrogen (e.g., one of the functional groups previously described) on the carbon atom adjacent to the acyl group. This amine group can be a secondary amine group, wherein the nitrogen atom bears, in addition to a hydrogen atom, a functional group (which can be any of the functional groups previously described) or a protecting group (i.e., a group capable of being removed and replaced by a hydrogen atom following a reaction of the acyl compound in which the protected amine group does not participate, e.g., an Fmoc group, a t-Boc group, a Cbz group or the like). The secondary amine group may, for example, bear a functional group having structure —CH(R¹)(R²) in which R¹ and R² are independently selected from the group consisting of hydrogen, hydrocarbyl, and substituted hydrocarbyl groups. The activated acyl group can be any derivative of a carboxyl group that is more susceptible to nucleophilic attack (specifically, to attack by a secondary amine) than a free carboxylic acid group or a methyl ester group. Illustrative examples of suitable activated acyl groups include acid fluorides, At esters, Bt esters, N-hydroxysuccinimide esters, pentafluorophenyl esters, O-acyl-ureas and the like. Any of the coupling agents known in the art of peptide coupling can be used to introduce an activated acyl group into the acyl compound (e.g., by conversion of a free carboxylic acid group) including, for example, HATU (2-(1H-7-azabenzotriazol-1-yl)-1,1,3,3-tetramethyl uranium hexafluorophosphate), BOP (benzotriazol-1-yl-oxy-tris(dimethylamino)phosphonium hexafluorophosphate), PyBOP (1H-1,2,3-benzotriazol-1-yloxy)-tris-(pyrrolidino)-phosphonium hexafluorophosphate), HBTU (O-benzotriazole-N,N,N′,N′-tetramethyl uranium hexafluorophosphate), N-hydroxybenzotriazole (HOBT), O-(1H-benzotriazole-1-yl)-N,N,N′,N′-tetramethyluronium tetrafluoroborate (TBTU), DCC (dicyclohexylcarbodiimide), DIC (diisopropylcarbodiimide), chloro-N,N,N′,N′-tetramethylformamidinium hexafluorophosphate or the like. Uronium and phosphonium salts of non-nucleophilic anions such as tetrafluoroborate or hexafluorophosphate are particularly useful. In one embodiment, an onium coupling agent is employed.

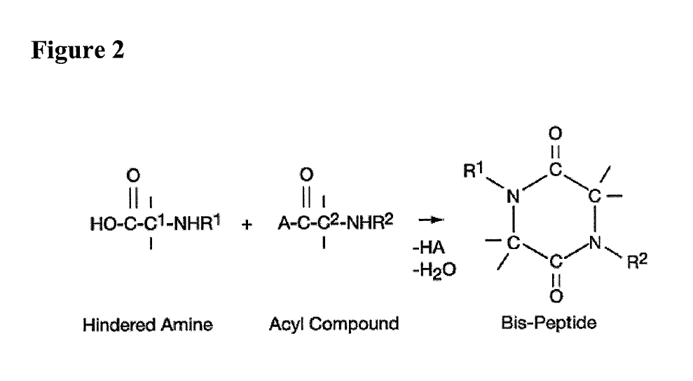

In one aspect, which is useful in the construction of bis-peptides, the acyl compound bears an amine group (which can be a secondary or protected amine group) alpha to the activated acyl group and the initial acylation yields an amide intermediate which undergoes dehydration and ring closure involving the amine group (if the amine was protected then it is first deprotected) of the acyl compound and the carboxylic acid group of the hindered amine to form a diketopiperazine ring. The dehydration may be facilitated by the use of a dehydrating agent such as a carbodiimide (e.g., diisopropylcarbodiimide, also known as DIC). This type of reaction is illustrated in FIG. 2, where R¹ and R² are the same or different and are independently selected from hydrocarbyl groups, substituted hydrocarbyl groups and protecting groups and A has the same meaning as in FIG. 1. One or both of R¹ and R² may form part of a ring structure which also includes the nitrogen atom of a secondary amine group and the carbon atom adjacent to that secondary amine group marked in FIG. 2 as C¹ or C².

In one embodiment, a functionalized bis-peptide is sequentially assembled in accordance with the following general procedure. A first building block (which can be attached to a resin or other support, if so desired) is selected which contains both a secondary amine group (where the amine nitrogen may be part of a ring structure, for example) and a free carboxylic acid group alpha to that secondary amine group (for example, the free carboxylic acid group may be attached to a carbon atom adjacent to the amine nitrogen atom, where the carbon atom is part of the same ring structure as the amine nitrogen atom). This first building block is reacted with a second building block, which contains a secondary amine group bearing a pendant functional group and an activated acyl group (e.g., an At ester) alpha to the secondary amine group as well as a protected amine group (e.g., NCbz) and a protected carboxylic acid group (e.g., —CO₂-tBu) alpha to the protected amine group. This reaction yields a bis-peptide containing a diketopiperazine ring formed by the interaction of the secondary amine group and free carboxylic acid group of the first building block with the secondary amine group and activated acyl group of the second building block, with the protected amine group and protected carboxylic acid group of the second building block remaining intact in the bis-peptide. If the diketopiperazine does not form quantitatively then dehydrating agent is added to assist diketopiperazine formation. These protecting groups are then removed to provide a secondary amine group and a free carboxylic acid group alpha to the secondary amine group, which are subsequently reacted similarly with a third building block which contains a secondary amine group bearing a pendant functional group (which can be different from the functional group in the second building block) and an activated acyl group alpha to the secondary amine group as well as a protected amine group and a protected carboxylic acid group alpha to the protected amine group. Similar cycles of reaction, deprotection and reaction with further building blocks may be repeated as desired to increase the length of the bis-peptide macromolecule and introduce different functional groups along the backbone. The stereochemistry and structure of the individual building blocks may be selected so as to vary and control the three-dimensional shape of the bis-peptide. The bis-peptide may be end-capped with various compounds to introduce further functionality at the terminus. For example, the secondary amine group and free carboxylic acid group alpha to the secondary amine group at the bis-peptide terminus can be reacted with a functionalized mono-amino acid to form a diketopiperazine ring at the terminus bearing a functional group attached to a carbon atom of the diketopiperazine ring.

In another embodiment, a functionalized bis-peptide is sequentially assembled in accordance with the following general procedure. A first building block (which an be attached to a resin or other solid support, if so desired) is selected which contains both a protected primary amine group (having the structure—NHPr, for example, where Pr is a protecting group such as Fmoc) and a protected carboxylic acid group alpha to that protected primary amine group (for example, the protected carboxylic acid group may be a —C(═O)ODmab group). The protected primary amine group is deprotected to provide a primary amine group, which is then functionalized using reductive amination (reaction with an aldehyde or ketone) or alkylation (reaction with an alkyl halide, for example) to convert the primary amine group to a secondary amine group bearing a functional group. The protected carboxylic acid group is then converted to an activated acyl group (for example, by deprotection of the carboxylic acid group and reaction of the resulting free carboxylic acid with a peptide coupling agent). This product is reacted with a second building block which contains a secondary amine group (which can be part of a ring structure, for example) and a free carboxylic acid group alpha to the secondary amine group as well as a protected primary amine group (e.g., NHFmoc) and a protected carboxylic acid group (e.g., —CO₂-Dmab) alpha to the protected primary amine group. This reaction yields a bis-peptide containing a diketopiperazine ring formed by the interaction of the functionalized secondary amine group and activated acyl group of the first building block with the secondary amine group and free carboxylic acid group of the second building block, with the protected primary amine group and protected carboxylic acid group of the second building block remaining intact in the bis-peptide. The protecting group on the protected primary amine group is then removed to provide a primary amine group, which is thereafter functionalized to introduce a functional group onto the nitrogen atom which is the same as or different from the first functional group incorporated into the bis-peptide. The protected carboxylic acid group is then deprotected to provide a free carboxylic acid group alpha to the functionalized secondary amine group. The bis-peptide is subsequently reacted with a third building block which contains a secondary amine group and a free carboxylic acid group alpha to the secondary amine group as well as a protected primary amine group and a protected carboxylic acid group alpha to the protected primary amine group. Similar cycles of deprotection, functionalization, activation of an acyl group and reaction with further building blocks may be repeated as desired to increase the length of the bis-peptide macromolecule and introduce different functional groups along the backbone. The stereochemistry and structure of the individual building blocks may be selected so as to vary and control the three-dimensional shape of the bis-peptide. The bis-peptide may be end-capped with various compounds to introduce further functionality at the terminus.

In still another embodiment of the invention, a bis-peptide is synthesized starting with a first building block (which may or may not be immobilized) that contains a functionalized secondary amine group (e.g., —NHR, where the nitrogen atom is not part of a ring structure and R is a functional group) and an activated acyl group alpha to the functionalized secondary amine group. This first building block is reacted with a second building block containing a secondary amine group (which can be part of a ring structure) and a free carboxylic acid group alpha to the secondary amine group as well as a functionalized secondary amine group (where the functional group may be the same as or different from the functional group in the first building block) and a protected carboxylic acid group (e.g., —CO₂Dmab) alpha to the functionalized secondary amine group. This reaction yields a bis-peptide containing a diketopiperazine ring formed by the interaction of the functionalized secondary amine group and activated acyl group of the first building block with the secondary amine group and free carboxylic acid group of the second building block, with the protected carboxylic acid group of the second building block remaining intact in the bis-peptide. The protected carboxylic acid group present in the bis-peptide may be converted to an activated acyl group and the bis-peptide further extended in a similar manner with a third building block containing a secondary amine group and a free carboxylic acid group alpha to the secondary amine group as well as a functionalized secondary amine group (where the functional group may be the same as or different from the functional group in the first and second building blocks) and a protected carboxylic acid group alpha to the functionalized secondary amine group. Additional cycles of reaction, deprotection, activation and reaction may be carried out with still more such building blocks to introduce different functional groups along the backbone and influence the three-dimensional shape of the bis-peptide. The bis-peptide may be end-capped with various compounds to introduce further functionality at the terminus.

The bis-peptides may be synthesized in solution using one or more suitable solvents. Solid-phase synthesis techniques may also be utilized, wherein a solid, insoluble resin or other support having functional groups (linkers) on which the bis-peptide can be built is employed. Suitable functional groups include, for example, amine groups (e.g., —NH₂) and hydroxyl groups (—OH). Aminomethyl polystyrene resins may be utilized. The bis-peptide remains covalently attached to the resin, which may, for example, be in the form of beads, until cleaved from the resin by a reagent such as trifluoro acetic acid. The bis-peptide is thus immobilized on the solid-phase resin during synthesis and can be retained on the resin during a filtration process, wherein liquid-phase reagents and soluble by-products of synthesis are flushed away. The general principle of solid-phase synthesis is one of repeated cycles of coupling-deprotection. That is, a first building block is attached to a resin such that the resin-attached building block contains a free primary or secondary amine group (in one embodiment, a primary amine group in the first building block, after being attached to the solid support is converted to a secondary amine group in which the nitrogen bears a functional group, e.g., a functional group —CH(R¹)(R²), using a reductive amination involving an alkyl halide or other suitable method). This amine group of the first building block containing an N-protected amine group as well as a carboxylic acid group (in one embodiment, an activated acyl group) to form an amide bond. The amine group of the second building block is then deprotected, revealing a new free amine group to which a further building block may be attached. The structures of the successive building blocks may be selected such that following formation of the initial amide bond, a second amide bond is formed between adjacent building blocks and a diketopiperazine ring is formed. Additionally, the building blocks employed may contain different functional groups attached to the secondary amine nitrogen of each building block, resulting in the production of an oligomeric bis-peptide having different pendant functional groups along its backbone, with the placement of the different functional groups being controlled as desired by the order in which the building blocks are reacted with the growing chains.

In solution phase synthesis, the development of optimized purification protocols for each intermediate requires a great deal of time. Solid state synthesis does not involve purification of intermediates, greatly accelerating the rate at which bis-peptides can be synthesized. Solution phase synthesis requires slightly lower quantities of building block because couplings are performed with stoichiometric amounts of monomers. However, the purified yields of bis-peptide, intermediates are generally 60-70%, so the savings do not present a compelling advantage for solution phase synthesis.

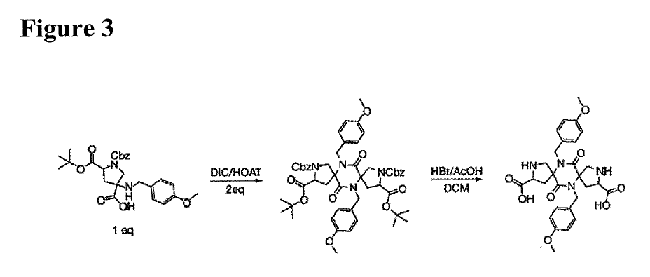

Hexasubstituted diketopiperazines may also be prepared in accordance with the present invention, either in solution or by means of solid state synthesis. FIG. 3 illustrates an example of a solution phase synthesis of a symmetric hexasubstituted diketopiperazine, where R may be any of the functional groups previously disclosed. Such hexasubstituted diketopiperazines may be suitably protected to also be incorporated into a bis-peptide oligomer or polymer or used as an independent scaffold.

TABLE 1
Linear and branched linkers (L) include the following functional
groups and combinations of the following functional groups
Linear linkers

| | |
|---|---|
| 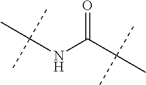 | Amide |
| 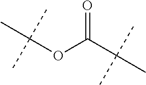 | Ester |
| 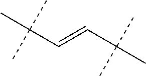 | cis or trans Alkene |
| 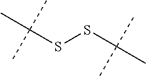 | Disulfide |
| 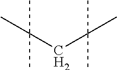 | Alkyl groups |
| 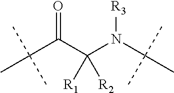 | Amino acids |
| 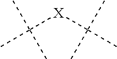 | ether, amine, tioether |
| 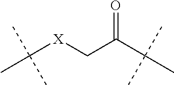 | acyl |
| 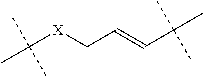 | allyl |
| 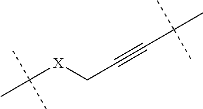 | propargyl |
| 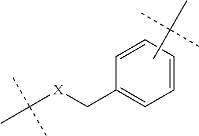 | benyl |
| 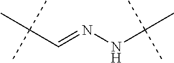 | Hydrazine |
| 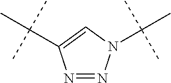 | Triazole |

Branched linkers include

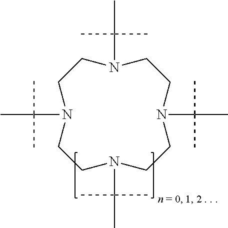

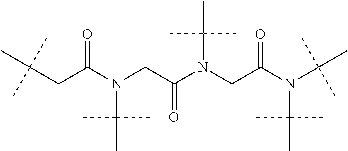

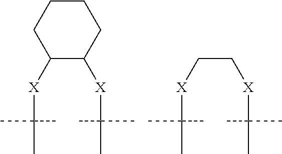

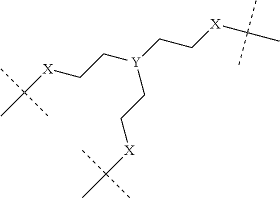

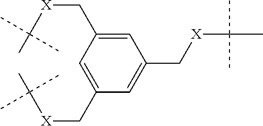

### Linear Assemblies:

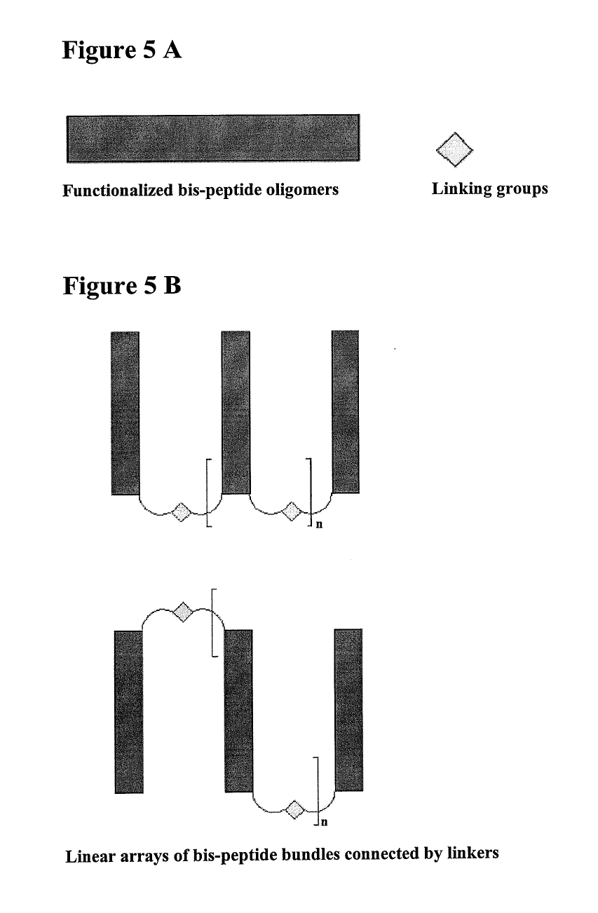

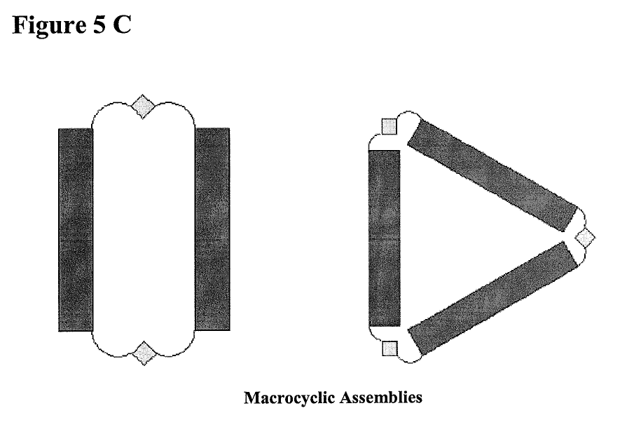

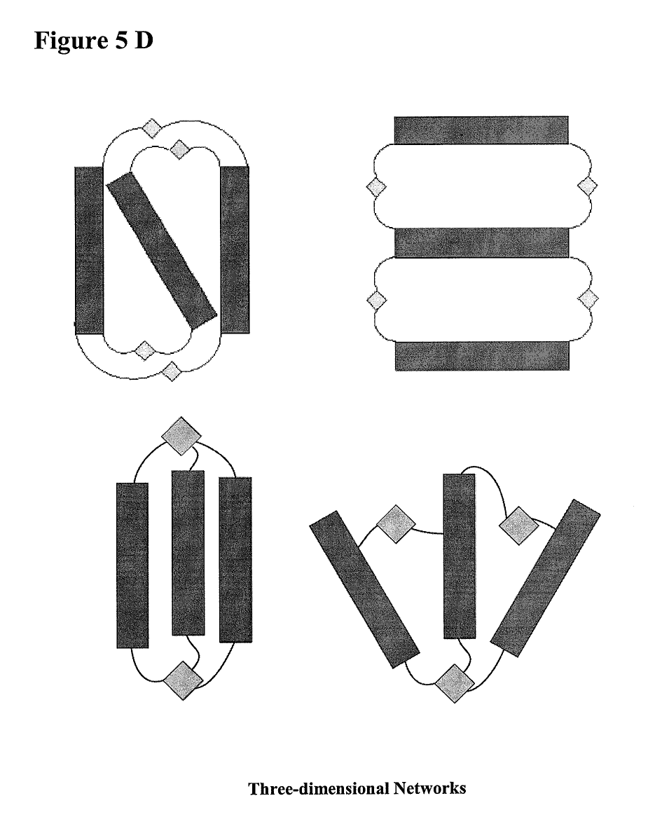

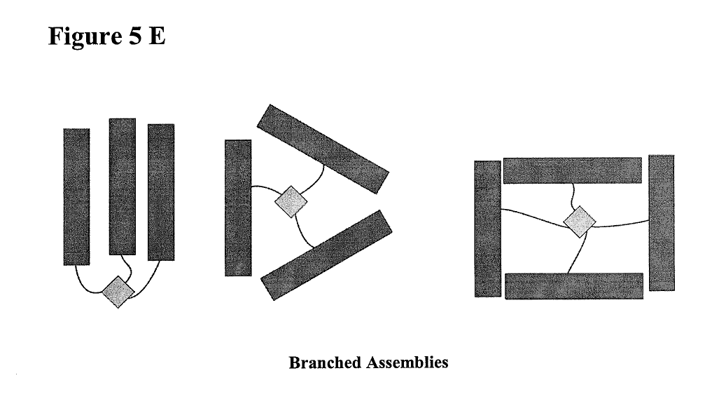

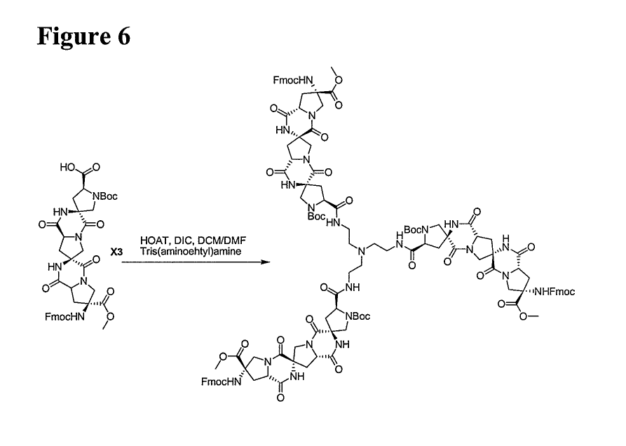

Functionalized bis-peptides can be joined in a variety of topologies. The schematic representations of FIG. 5 illustrate certain functionalized bis-peptide oligomers as shaded rectangles. They are joined by lines to linking groups that are indicated by the diamond symbols.

The reactive groups that combine to form the linkers can be located anywhere on the bis-peptide. For example, the reactive groups can be located on opposite ends of the bis-peptide segment. One reactive group can be located in the interior of the bis-peptide segment and one reactive group can be located on an end of the bis-peptide segment. Both can be located in the interior of the bis-peptide segment. Both functional groups can be located on the same end of a bis-peptide segment. Covalent bis-peptide bundles can be further functionalized on their ends or interiors.

A sequential assembly approach may be utilized to synthesize linear arrays of bis peptides containing two or more functionalized bis-peptides, containing any combination of functionalized bis-peptides. Linear bis-peptide arrays can be synthesized in solution phase by replacing the resin bound linker with a suitable orthogonal protecting group. The bis-peptides can be combined using any of a variety of linking chemistries including amides, esters, secondary amines, triazoles, rings using Diels-Alder couplings, hydrazones, hydrazides, disulphide linkages, alkyl-chains, aromatic rings, olefins formed using reactions including metathesis, phosphonate ester linkages, silyl linkages, organometallic linkages and alkyl chain linkages and combinations of these to join bis-peptides into linear arrays. Bis-peptides containing complementary reactive groups may be polymerized into very large polymers and block-copolymers of functionalized bis-peptides.

These linear bis-peptide arrays can be derivatized with additional functional groups including those described supra (see “Functional groups include”). The linear bis-peptide arrays can also be derivatized with other functionalized bis-peptides, peptides, carbohydrates, DNA, RNA and small molecules.

Besides linear arrays, functionalized bis-peptides may be conjugated onto cyclic, linear and branched scaffolds such as azacrowns, polyamines and dendrimer molecules.

### Macrocyclic Assemblies:

Linear arrays of bis-peptides can be functionalized with complementary reactive groups on their extreme ends and linked together to form macrocycles. Macrocycles containing two, three and more functionalized bis-peptides can be formed by this method.

Linear arrays of bis-peptides can be functionalized to display multiple reactive groups within their bis-peptide segments or on their ends and then induced to cross-react selectively either all at once or over two or more sequential steps. The rigid, pre-organized nature of bis-peptides allows one to display these reactive groups in such a way that they can only cross-react with their intended partners to create precise three-dimensional networks. Such covalent bis-peptide networks like these can be synthesized using a variety of linking groups, containing a variety of bis-peptides with different stereochemistry, functional groups and numbers of monomers.

### Three-Dimensional Networks

Three-dimensional networks or covalent bundles, such as those illustrated in FIG. 5 D can be assembled with combinations of diverse functionalized bis-peptides of different length, different stereochemistry and different functional group display. These bis-peptide macrocycles and covalent bundles can be further derivatized with additional functional groups including those described supra (see “Functional groups include”) as well as with functionalized bis-peptides, peptides, carbohydrates, DNA, RNA and small molecules. The functionalized bis-peptide segments may be linked using different linking chemistry including amides, esters, secondary amines, triazoles, rings using Diels-Alder couplings, hydrazones, hydrazides, disulphide linkages, alkyl-chains, aromatic rings, olefins formed using metathesis, phosphonate ester linkages, silyl linkages, organometallic linkages and alkyl chain linkages and combinations of these. Covalent bis-peptide bundles may be functionalized so that they self-assemble into larger covalent complexes and materials via multiple covalent and non-covalent linkages.

### EXAMPLES

In the examples and schemes that follow, the general bis-peptide segment of Formula I was utilized to prepare bis-peptide arrays, wherein Rx is a protecting group.

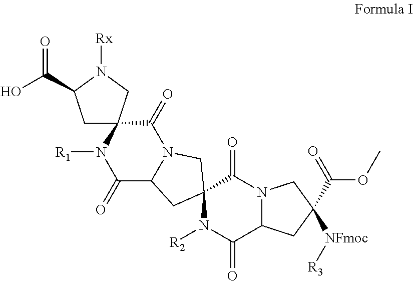

In Examples 1-6 and the corresponding schemes included in those examples, each of R₁, R₂, R₃ etc. was hydrogen, and all stereocenters were set to (S). However, it may be appreciated that any combination functional groups R₁, R₂, R₃, etc. with any combination of (S) and (R) stereochemistry may be utilized, using similar synthetic routes. Example 7 is an example of a functionalized bis-peptide macrocycle.

Unless otherwise indicated, in all the schemes that follow, the conditions indicated by lowercase were as follows: a) 1:19 Piperidine/DMF; b) Fmoc-Nal-OH, HATU, DIPEA, NMP; c) Bis-Peptide Oliogmer (Formula I), PyAOP, DIPEA, NMP; d) Fmoc-D-Dab(ivDde)-OH; e) (PPh₃)₄Pd, BH₃:DMA, DCM; f) Boc-Gly-OH, HATU, DIPEA, NMP; g) 1:49 Hydrazine/DMF; h) Fmoc-Dab(Fmoc)-OH, HATU, DIPEA, NMP; j) Bromoacetic Anhydride, DIPEA, NMP; k) 38:1:1 TFA/H₂O/TIPS; m) 1:99 TEA/DMF (0.0015M); n) (i) 1:19 piperidine/DMF; (ii) Fmoc-Dab(Boc)-OH, HATU, DIPEA, NMP; o) Fmoc-Gly-OH, HATU, DIPEA, NMP.

### Reaction Procedures:

All bis-amino acids were synthesized according to established literature procedures. All amino acids including diamines were purchased from either Novabiochem or Bachem. O-(7-Azabenzotriazole-1-yl)-N,N,N′N′-tetramethyluronium hexafluorophosphate (HATU) and 1-Hydroxy-7-azabenzotriazole (HOAT) were purchased from Genscript. All other reagents were purchased from Aldrich. Flash Chromatography was performed on an ISCO CombiFlash Rf with ISCO prepackaged silica gel or C18 columns. Analytical HPLC-MS analysis was performed on a Agilent Series 1200 HPLC attached to an Agilent Single Quadrole ESI Mass Spec with a Waters Xterra MS C18 column (3.5 um packing, 4.6 mm×150 mm) with a solvent system of water/acetonitrile with 0.1% formic acid at a flow rate of 0.8 mL/min. Preparatory Scale HPLC purification was performed on a Agilent Series 1100 HPLC with a Waters Xterra column (5 um packing, 7.8 mm×150 mm) with a solvent system of water/acetonitrile with 0.1% formic acid at a flow rate of 3 mL/min.

In all the examples that follow, the procedures indicated by upper-case letters A, B, etc. were as follows:

### Procedure (A): Attachment to Trityl Resin

To a solution of trityl resin and the amino acid (0.9 equivalents based on resin loading) in DCM (10 mL/g of resin) was added DIPEA (4 equivalents based on resin loading). The reaction mixture was stirred overnight. The solution was poured through a solid phase reactor to remove the resin from solution and washed with DMF, DCM, DMF, IPA, DMF, DCM and DMF.

### Procedure (B): Attachment to HMBA Resin

To a solution of amino acid (3 equivalents based on resin loading) and MSNT (3 equivalents based on resin loading) was added MeIm (2.25 equivalents based on resin loading in DCM (5 mL/mmole of amino acid). The reaction mixture was agitated for 5 minutes then added to a pre-swelled (with DMF) portion of resin in a solid phase reactor and stirred for 4 hours. The resin was filtered and washed with DMF, DCM, DMF, IPA, DMF, DCM and DMF.

### Procedure (C): Attachment to NovaPEG Rink Amide Resin

To a solution of amino acid (3 equivalents based on resin loading) and HATU (3 equivalents based on resin loading in NMP (5 mL/mmole of amino acid) was added DIPEA (6 equivalents based on resin loading). The reaction mixture was agitated for 5 minutes then added to a pre-swelled (with DMF) portion of resin in a solid phase reactor and stirred for 4 hours. The resin was filtered and washed with DMF, DCM, DMF, IPA, DMF, DCM and DMF.

### Procedure (D): Coupling of Functionalized Bis-Amino Acid

To a solution of the functionalized bis-amino acid (3 equivalents relative to resin loading) and HOAT (6 equivalents relative to bis-amino acid) in 1:2 DMF/DCM (18 mL/mmole) was added DIC (1 equivalent relative to bis-amino acid). The reaction was stirred for 90 minutes then added to a pre-swelled (with DMF) portion of resin in a solid phase reactor. A solution of DIPEA (2 equivalents based on resin loading) in DMF (6 mL/mmole) and stirred for 3 hours. An additional aliquot of DIC (3 equivalents relative to resin loading) was added and stirred for 1 hour. The resin was filtered and washed with DMF, DCM, DMF, IPA, DMF, DCM and DMF.

### Procedure (E): Solution Coupling of Functionalized Bis-Amino Acid

To a solution of the functionalized bis-amino acid (1 equivalent) and HOAT (6 equivalents) in 1:2 DMF/DCM (18 mL/mmole) was added DIC (1 equivalent). The reaction was stirred for 90 minutes then added to a second bis-amino acid (0.8 equivalents) and DIPEA (1.6 equivalents) in DMF (6 mL/mmole). The reaction was stirred for 7.5 hours and an additional aliquiot of DIC (2 equivalents was added and stirred overnight. The reaction was diluted with EtOAc then washed with sat. NH₄Cl (aq)×3, sat. NaCl (aq)×1, sat. NaHCO₃ (aq)×3, NaCl (aq)×2, dried over Na₂SO₄, filtered, and concentrated under reduced pressure.

### Procedure (F): HATU Coupling

To a solution of amino acid (3 equivalents based on resin loading) and HATU (3 equivalents based on resin loading in NMP (5 mL/mmole of amino acid) was added DIPEA (6 equivalents based on resin loading). The reaction mixture was agitated for 5 minutes then added to a pre-swelled (with DMF) portion of resin in a solid phase reactor and stirred for 4 hours. The resin was filtered and washed with DMF, DCM, DMF, IPA, DMF, DCM and DMF.

### Procedure (G): PyAOP Coupling

To a solution of amino acid (3 equivalents based on resin loading) and PyAOP (3 equivalents based on resin loading in NMP (5 mL/mmole of amino acid) was added DIPEA (6 equivalents based on resin loading). The reaction mixture was agitated for 5 minutes then added to a pre-swelled (with DMF) portion of resin in a solid phase reactor and stirred for 12 hours. The resin was filtered and washed with DMF, DCM, DMF, IPA, DMF, DCM and DMF.

### Procedure (H): Boc and Tert-Butyl Ester Deprotection

A solution of 5% TIPS in TFA (10 mL/mmole based on resin loading) was added to a pre-swelled (with DMF) portion of resin in a solid phase reactor and stirred for 30 minutes. The resin was filtered and washed with DMF, DCM, DMF, IPA, DMF, DCM and DMF. This process was repeated in duplicate to ensure complete deprotection.

### Procedure (I): Cbz and Tert-Butyl Ester Deprotection

A solution of the Bis-peptide oligomer and 1:2 HBr/AcOH (5 mL/mmole) in DCM (5 mL/mmole) was stirred for 4 hours. The reaction mixture was concentrated under reduced pressure.

### Procedure (J): Fmoc Deprotection

A solution of 20% of piperidine in DMF (10 mL/mmole based on resin loading) was added to a pre-swelled (with DMF) portion of resin in a solid phase reactor and stirred for 15 minutes. The resin was filtered and washed with DMF, DCM, DMF, IPA, DMF, DCM and DMF. This process was repeated in duplicate to ensure complete deprotection.

### Procedure (K): Alloc Deprotection

A solution of borane:dimethylamine complex (6 equivalents based on resin loading) in DCM (10 mL/mmole based on resin loading) was added to a pre-swelled (with DMF) portion of resin in a solid phase reactor and stirred for 5 minutes. To this solution was added a solution of tetrakis(triphenylphosphine)palladium(0) (0.1 equivalents based on resin loading) in DCM (10 mL/mmole based on resin loading). The reaction mixture was stirred for 2 hour. The resin was filtered and washed with DMF, DCM, DMF, IPA, DMF, DCM and DMF.

### Procedure (L): IvDde Deprotection

A solution of 2% of hydrazine in DMF (10 mL/mmole based on resin loading) was added to a pre-swelled (with DMF) portion of resin in a solid phase reactor and stirred for 20 minutes. The resin was filtered and washed with DMF, DCM, DMF, IPA, DMF, DCM and DMF. This process was repeated in triplicate to ensure complete deprotection.

### Procedure (M): Bromoacetate Acylation

To a solution of bromoacetic anhydride (3 equivalents based on resin loading) in NMP (5 mL/mmole of amino acid) was added DIPEA (6 equivalents based on resin loading). The reaction mixture was agitated for 5 minutes then added to a pre-swelled (with DMF) portion of resin in a solid phase reactor and stirred for 4 hours. The resin was filtered and washed with DMF, DCM, DMF, DCM, DMF, DCM and DMF

### Procedure (N): Alloc Protection

To a solution of the oligomer (1 equivalent) and Alloc-Cl (1.1 equivalents) in DCM (10 mL/mmole) was added DIPEA (3 equivalents). The reaction mixture was stirred overnight then diluted with EtOAc. The solution was washed with sat. NH₄Cl (aq)×3, sat. NaCl (aq)×2, dried over Na₂SO₄, filtered, and concentrated under reduced pressure.

Procedure (O): Safety Catch Cleavage from HMBA Resin

A solution of 10% DIPEA in DMF (10 mL/mmole based on resin loading was added to a portion of resin and stirred overnight. The resin was filtered. The filtrate was concentrated, reconstituted in 50% MeCN in water (0.1% formic acid) and freeze-dried.

Procedure (P): Liberation from Trityl or Rink Amide Solid Phase Resins

A solution of 5% TIPS and 5% water in TFA (20 mL/mmole based on resin loading was added to a portion of resin (successively washed with DCM and MeOH, and thoroughly dried under vacuum) and stirred for 4 hours. The resin was filtered and rinsed with TFA. The filtrate was concentrated, reconstituted in 50% MeCN in water (0.1% formic acid) and freeze-dried.

### Procedure (Q): Rigidification of Bis-Peptide Oligomers:

A solution of the bis-peptide oligomer in 0.5 M NH₄OAc in MeCN/water 1:1 (20 mL/μmole) was heated to 60° C. and stirred overnight and freeze-dried.

### Procedure (R): Cross-Linking in Aqueous Solution:

A solution of the bis-peptide linear assembly in 50% MeCN in water (22 mL/μmole) was added dropwise to a solution of 0.05M pH 7 Phosphate Buffer (44 ml/μmole) and stirred for 60 hours and freeze-dried.

### Procedure (S): Cross-Linking in Organic Solution:

A solution of the bis-peptide linear assembly in DMF (22 mL/μmole) was added dropwise to a solution of 1.5% TEA in DMF (44 mL/μmole) and stirred overnight. The solution was added dropwise to a solution of diethyl ether (666 mL/μmole, chilled to −20° C.) and centrifuged. The solvent was decanted and the pellet was reconstituted in 50% MeCN in water (0.1% formic acid) and freeze-dried.

### Preparative Example 1

Solid Phase Synthesis of Non-Functionalized Bis-Peptides

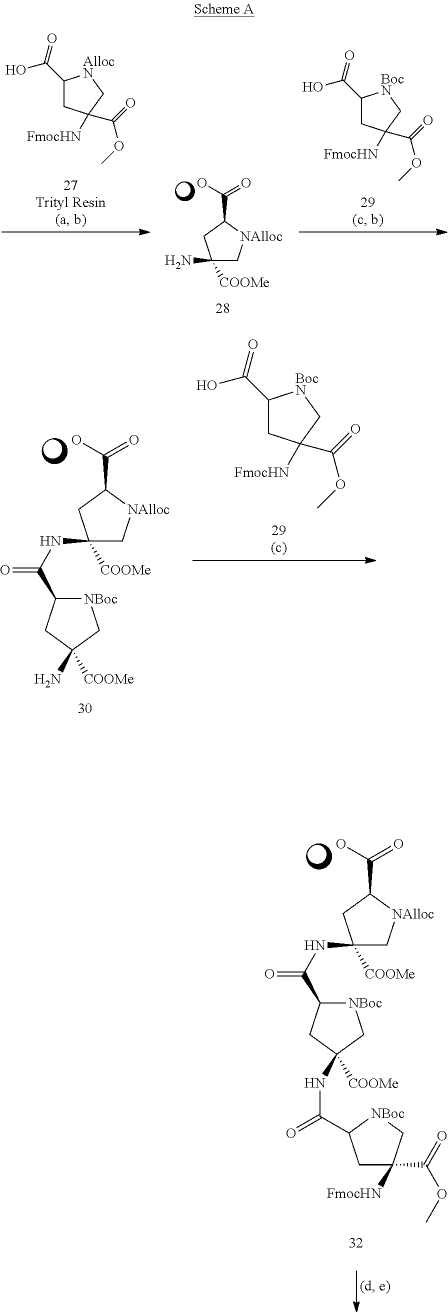

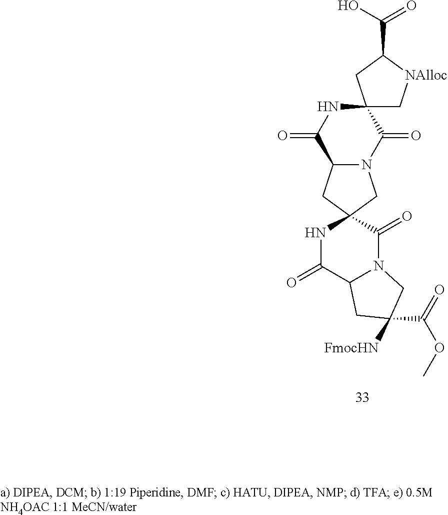

Compound 27 (494.5 mg, 1 mmole) was attached to Trityl resin (1 g, 1.1 mmole) according to procedure (A) using DCM (10 mL) and DIPEA (696.8 μL, 4 mmoles). The terminal Fmoc group was removed according to procedure (J) using 20% piperidine in DMF (900 μL).

Compound 29 (1.53 g, 3 mmole) was coupled according to procedure (F) using HATU (1.14 g, 3 mmoles), NMP (15 mL), and DIPEA (1.05 mL, 6 mmoles). The terminal Fmoc group was removed according to procedure (J) using 20% piperidine in DMF (900 μL).

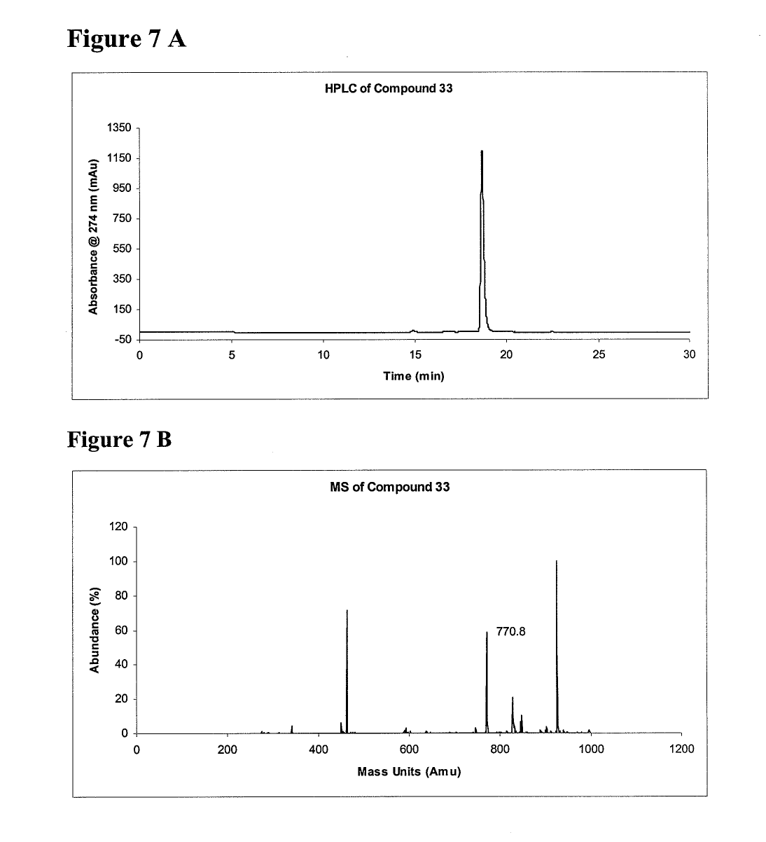

Compound 29 (1.53 g, 3 mmole) was coupled according to procedure (F) using HATU (1.14 g, 3 mmoles), NMP (15 mL), and DIPEA (1.05 mL, 6 mmoles). The oligomer was liberated from the resin according to procedure (P) using 95% TFA/2.5% TIPS/2.5% Water (20 mL). The oligomer was rigidified according to procedure (Q) using 0.5 M NH4OAc in MeCN/water 1:1 (20 mL) to yield 33. Compound 33 corresponds to a compound of Formula I where R₁, R₂ and R₃ are all hydrogens and Rx is Alloc. HPLC and MS characterization of compound 33 is shown in FIG. 7.

### Preparative Example 2

### Solid Phase Synthesis of Functionalized Bis-Peptides

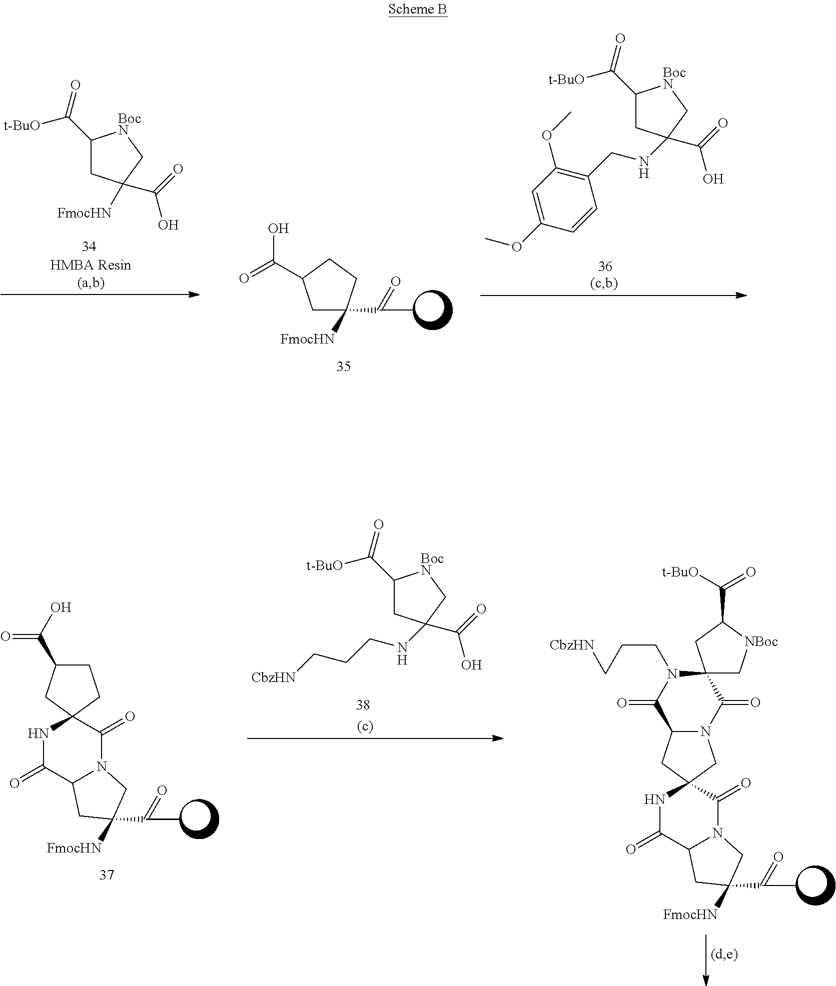

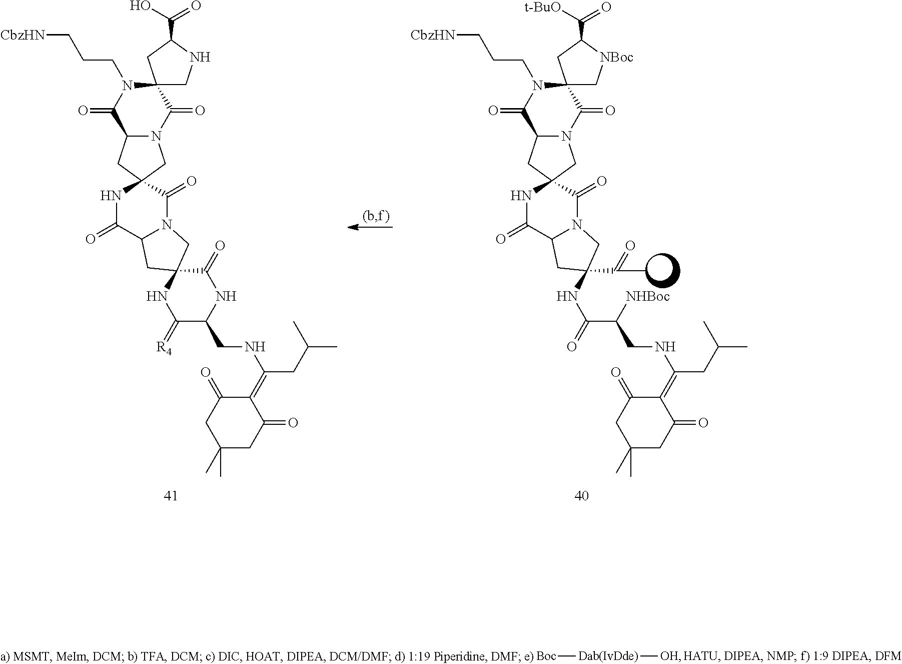

Compound 34 (165.8 mg, 300 μmoles) was attached to HMBA resin (113.6 mg, 100 μmoles) according to procedure (B) using MSNT (88.9 mg, 300 μmoles), DCM (1.5 mL) and MeIm (17.9 μL, 225 μmoles). The terminal Boc and tert-Butyl ester were removed according to procedure (H) using 5% TIPS in TFA (imp.

Compound 36 (144.2 mg, 300 μmoles) was coupled according to procedure (D) using HOAT (245.0 mg, 1.8 mmoles), 1:2 DMF/DCM (5.4 mL), and DIC (46.8 μL, 300 μmoles). The additions of DIPEA (348.4 μL, 200 μmoles) in DMF (1.8 mL) and DIC (140.9 μL, 900 μmoles) were added at the desired times. The terminal Boc and tert-Butyl ester were removed according to procedure (H) using 5% TIPS in TFA (1 mL).

Compound 38 (170.9 mg, 300 μmoles was coupled according to procedure (D) using HOAT (245.0 mg, 1.8 mmoles), 1:2 DMF/DCM (5.4 mL), and DIC (46.8 μL, 300 μmoles). The additions of DIPEA (348.4 μL, 200 μmoles) in DMF (1.8 mL) and DIC (140.9 μL, 900 μmoles) were added at the desired times. The terminal Fmoc group was removed according to procedure (J) using 20% piperidine in DMF (900 μL).

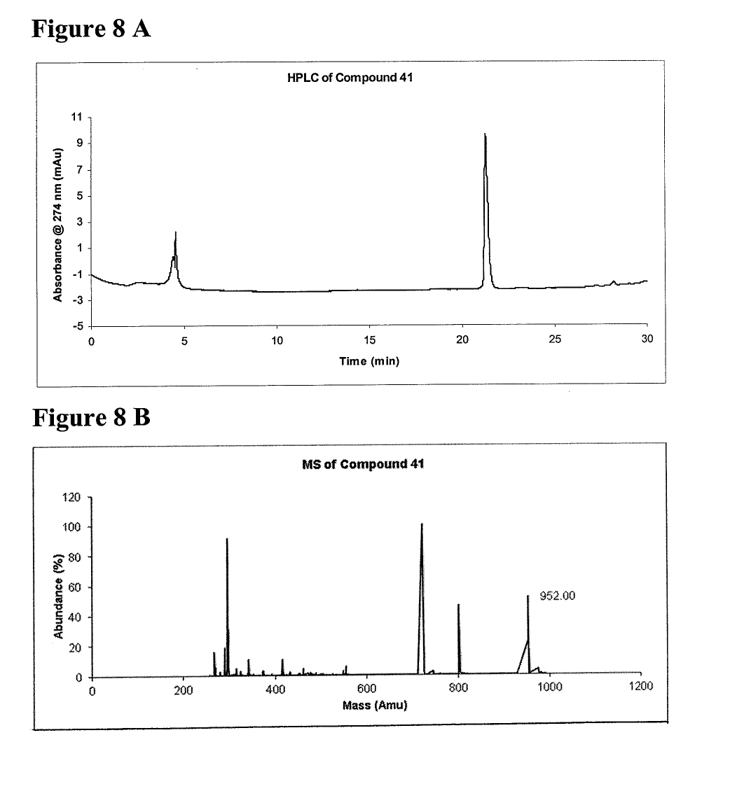

Boc-Dab(IvDde)-OH (141.9 mg, 300 μmoles) was coupled according to procedure (F) using HATU (114.1 mg, 300 μmoles), NMP (1.5 mL), and DIPEA (104.5 μL, 600 μmoles). The terminal Boc and tert-Butyl ester were removed according to procedure (H) using 5% TIPS in TFA (1 mL). The oligomer 41 was liberated from the resin according to procedure (O) using 10% DIPEA in DMF (3 mL). The reverse phase C18 HPLC-MS characterization of compound 41 is shown in FIG. 8.

### Preparative Example 3

### Solution Synthesis of Functionalized Bis-Peptides

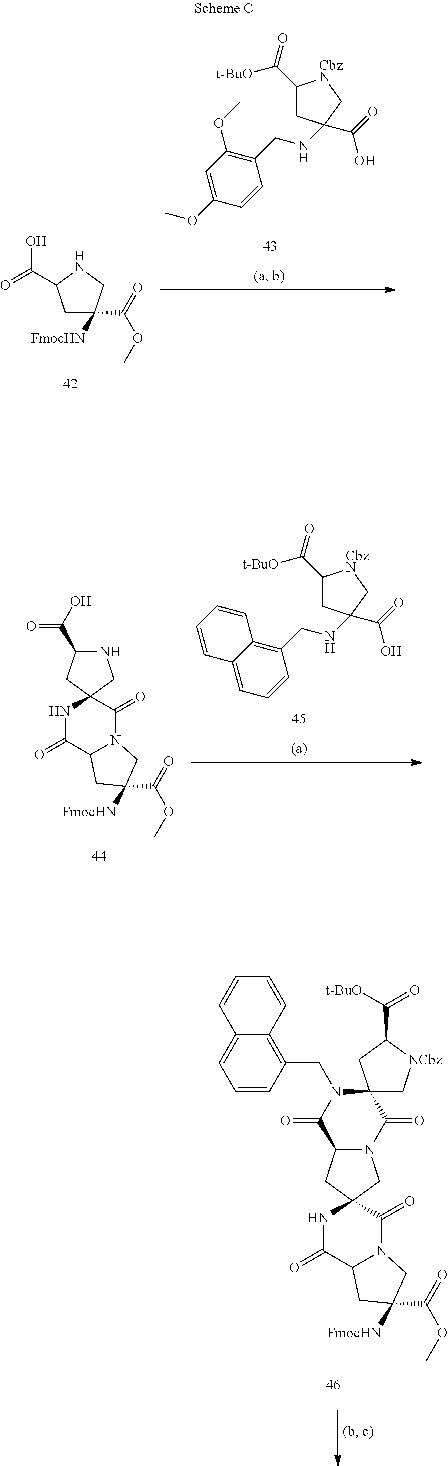

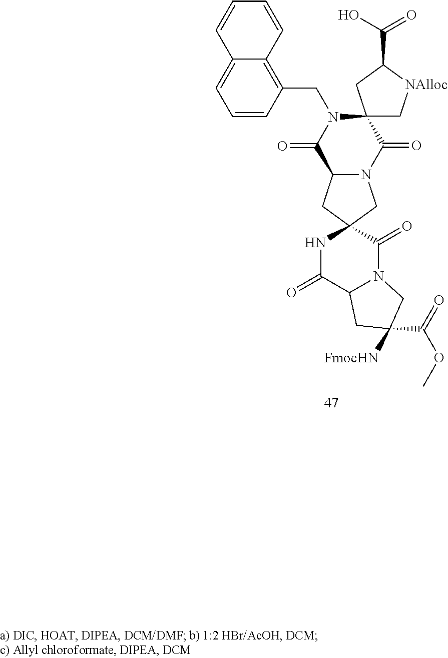

Compound 43 (144.2 mg, 300 μmoles) was coupled according to procedure (E) using HOAT (245.0 mg, 1.8 mmoles), 1:2 DMF/DCM (5.4 mL), and DIC (46.8 μL, 300 μmoles). This reaction mixture was added to compound 42 (98.5 mg, 240 μmoles) and DIPEA (83.6 μL, 480 μmoles) in DMF (1.8 mL) and DIC (94.0 μL, 600 μmoles) was added at the desired time. The terminal Cbz and tert-Butyl ester were removed according to procedure (I) using 1:2 HBr/AcOH (1.5 mL) in DCM (1.5 mL).

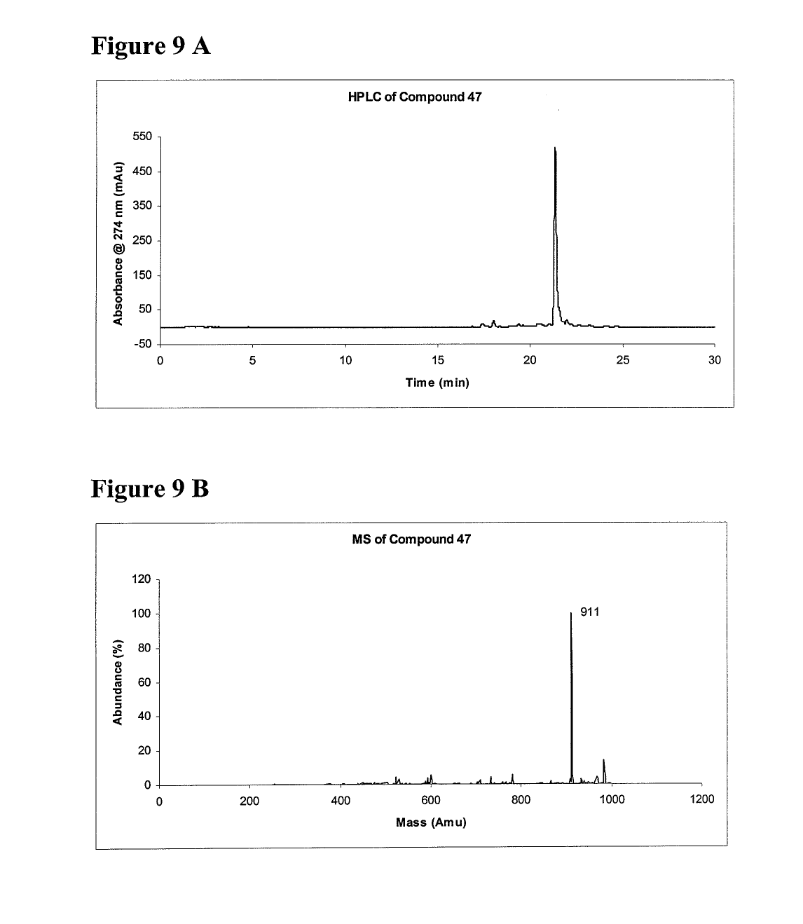

Compound 45 (144.2 mg, 300 μmoles) was coupled according to procedure (E) using HOAT (245.0 mg, 1.8 mmoles), 1:2 DMF/DCM (5.4 mL), and DIC (46.8 μL, 300 μmoles). This reaction mixture was added to Dimer from above (131.6 mg, 240 μmoles) and DIPEA (83.6 μL, 480 μmoles) in DMF (1.8 mL) and DIC (600 μmoles) was added at the desired time. The terminal Cbz and tert-Butyl ester were removed according to procedure (I) using 1:2 HBr/AcOH (1.5 mL) in DCM (1.5 mL). The trimer 47 was Alloc protected according to procedure (N) using Alloc-Cl (28.1 μL, 264 μmoles), DCM (3 mL), and DIPEA (125.4 μL, 720 μmoles) to yield compound 47. The reverse phase C18 HPLC-MS characterization of compound 47 is shown in FIG. 9.

### Example 1

### Synthesis of a Linear Array Containing Two Bis-Peptides

The two-bis-peptide linear array 5 was prepared according to Scheme 1 as follows.

NovaPEG Rink Amide Resin (81.1 mg, 30 μmoles loading) was placed in an 8 mL solid phase reactor. (S)—N-Fmoc-1-Naphthylalanine-OH (39.4 mg, 90 μmoles) was attached according to procedure (C) using HATU (34.2 mg, 90 μmoles), NMP (450 μL) and DIPEA (31.4 μL, 180 μmoles). The terminal Fmoc group was removed according to procedure (J) using 20% piperidine in DMF (900 μL).

Compound 33 (69.4 mg, 90 μmoles) was coupled according to procedure (G) using PyAOP (46.9 mg, 90 μmoles), NMP (450 μL), and DIPEA (31.4 μL, 180 μmoles). The terminal Fmoc group was removed according to procedure (J) using 20% piperidine in DMF (900 μL).

Fmoc-D-Dab(IvDde)-OH (49.2 mg, 90 μmoles) was coupled according to procedure (F) using HATU (34.2 mg, 90 μmoles), NMP (450 μL) and DIPEA (31.4 μL, 180 μmoles). The terminal Fmoc group was removed according to procedure (J) using 20% piperidine in DMF (900 μL). Exposure to the deprotection solution was extended to 2 hours to enable complete diketopiperazine closure.

The Alloc group was removed according to procedure (K) using borane:dimethylamine complex (10.6 mg, 180 μmoles) in DCM (450 μL) and tetrakis(triphenylphosiphine)palladium(0) (10.4 mg, 9 μmoles) in DCM (45 μL). Boc-Gly-OH (15.8 mg, 450 μmoles) was coupled according to procedure (C) using HATU (34.2 mg, 90 μmoles), NMP (450 μL), and DIPEA (31.4 μL, 180 μmoles). The terminal IvDde group was removed according to procedure (L) using 2% Hydrazine in DMF (900 μL).

Compound 33 (69.4 mg, 90 μmoles) was coupled according to procedure (G) using PyAOP (46.9 mg, 90 μmoles), NMP (450 μL), and DIPEA (31.4 μL, 180 μmoles). The terminal Fmoc group was removed according to procedure (J) using 20% piperidine in DMF (900 μL).

Fmoc-L-Dab(Fmoc)-OH (50.6 mg, 90 μmoles) was coupled according to procedure (F) using HATU (34.2 mg, 90 μmoles), NMP (450 μL) and DIPEA (31.4 μL, 180 μmoles). The terminal Fmoc group was removed according to procedure (J) using 20% piperidine in DMF (900 μL). Exposure to the deprotection solution was extended to 2 hours to enable complete diketopiperazine closure, to form the resin bound compound 5. This resin bound oligomer of two bis-peptides contains two bis-peptides joined by a flexible alkyl-amide linker.

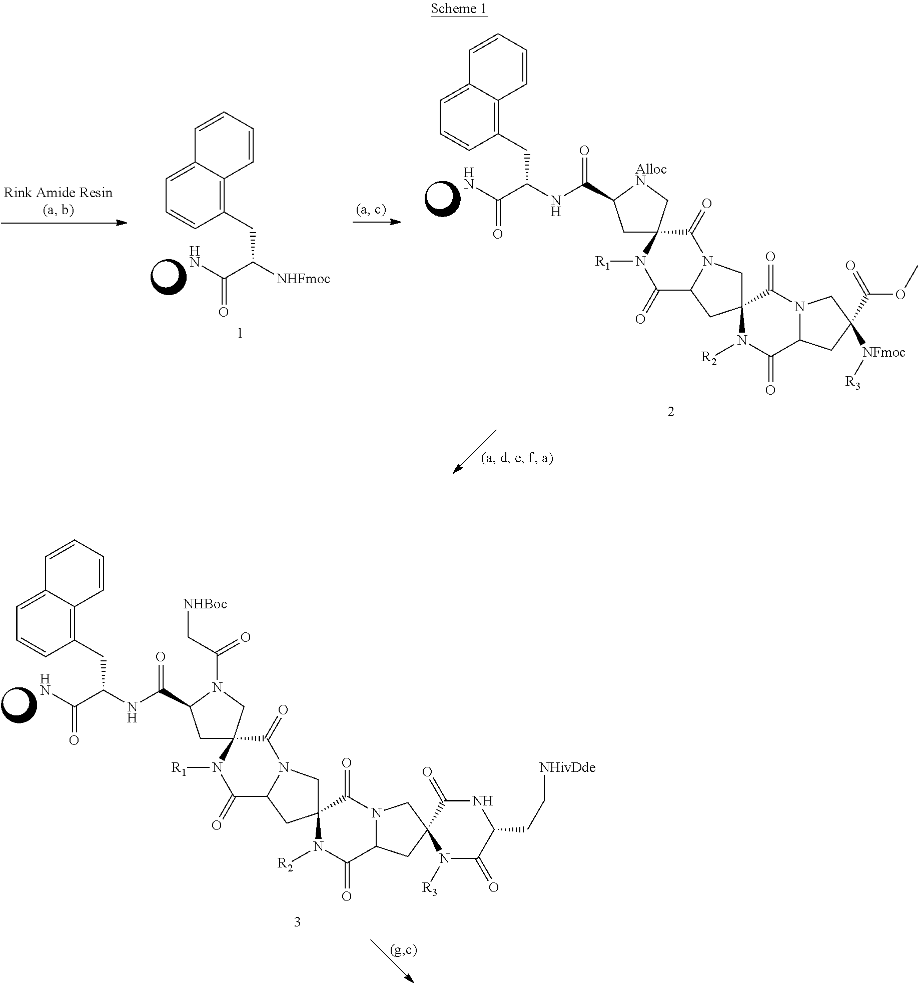

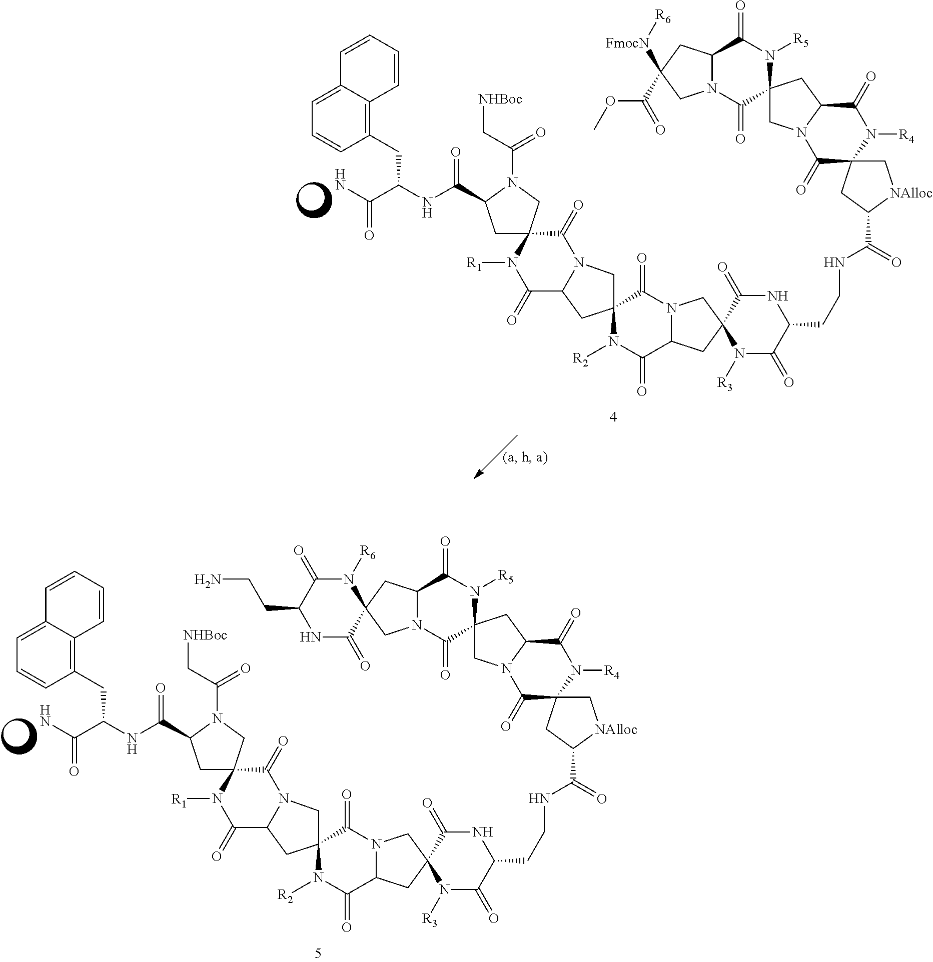

### Example 2

### Functionalized Linear Array Containing Two Bis-Peptides

The two-bis-peptide linear array 5 was further functionalized on resin by reacting the free primary amine with bromoacetic anhydride to incorporate an electrophilic bromoacetate on the leading end. Bromoacetate was introduced according to procedure (M) using bromoacetic anhydride (23.4 mg, 90 μmoles), NMP (450 μL), and DIPEA (31.4 μL, 180 μmoles). The linear assembly of bis-peptides was liberated from the resin according to procedure (P) using 95% TFA/2.5% Tips/2.5% Water (1.8 mL) to yield 6, as shown in Scheme 2.

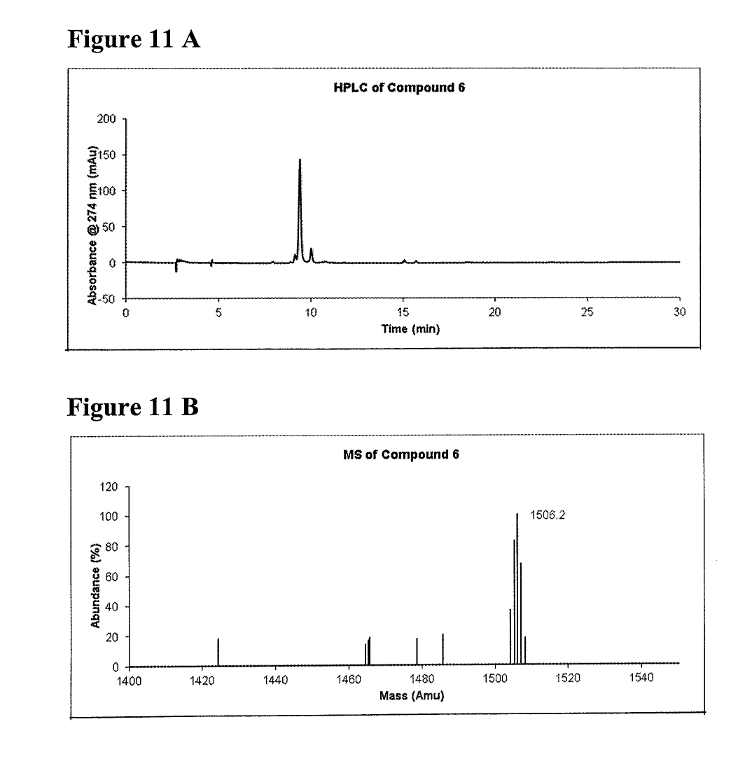

The reverse phase C18 HPLC-MS characterization of compound 6 where R₁ through R₆ are hydrogen is provided in FIG. 11.

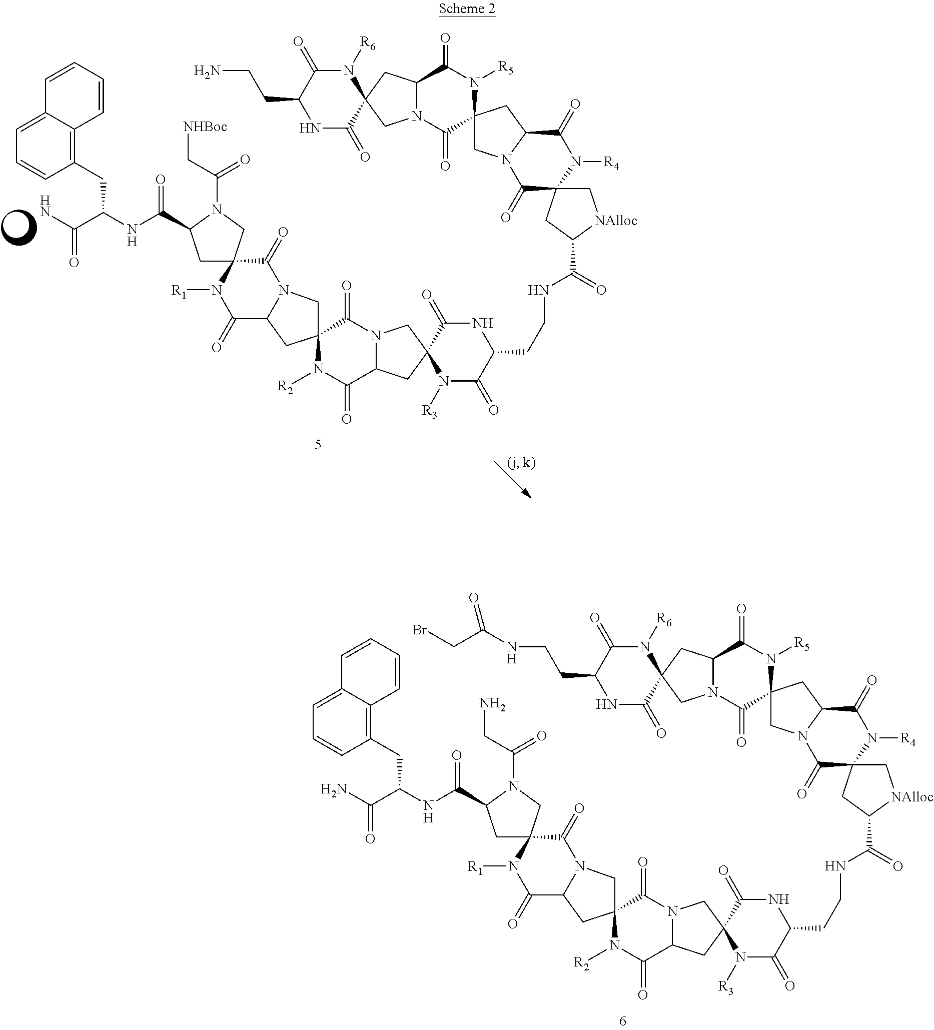

### Example 3

### Larger Functionalized Bis-Peptide Linear Array

To demonstrate that larger functionalized bis-peptide linear arrays can be created compound 5 was extended it with an additional functionalized bis peptide to create 7—a linear array of three functionalized bis-peptides.

Compound 33 (69.4 mg, 90 μmoles) was coupled to 5 according to procedure (G) using PyAOP (46.9 mg, 90 μmoles), NMP (450 μL), and DIPEA (31.4 μL, 180 μmoles). The terminal Fmoc group was removed according to procedure (J) using 20% piperidine in DMF (900 μL).

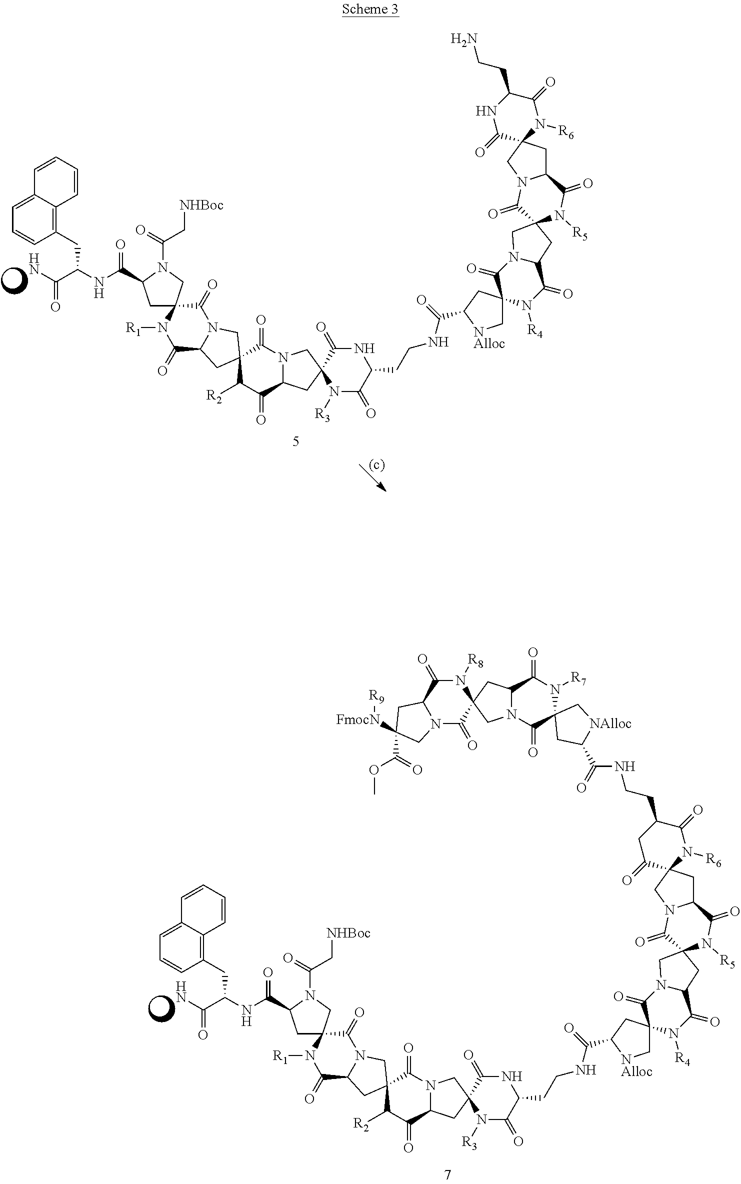

Compound 7 was further extended by removal of the leading end Fmoc group and coupling of a bis-Fmoc protected diaminobutanoic acid through a diketopiperazine linkage, as shown in Scheme 4.

Fmoc-L-Dab(Fmoc)-OH (50.6 mg, 90 μmoles) was coupled to 7 according to procedure (F) using HATU (34.2 mg, 90 μmoles), NMP (450 μL) and DIPEA (31.4 μL, 180 μmoles). The terminal Fmoc group was removed according to procedure (J) using 20% piperidine in DMF (900 μL). Exposure to the deprotection solution was extended to 2 hours to enable complete diketopiperazine closure to produce compound 8 where R₁ to R₉ are all H.

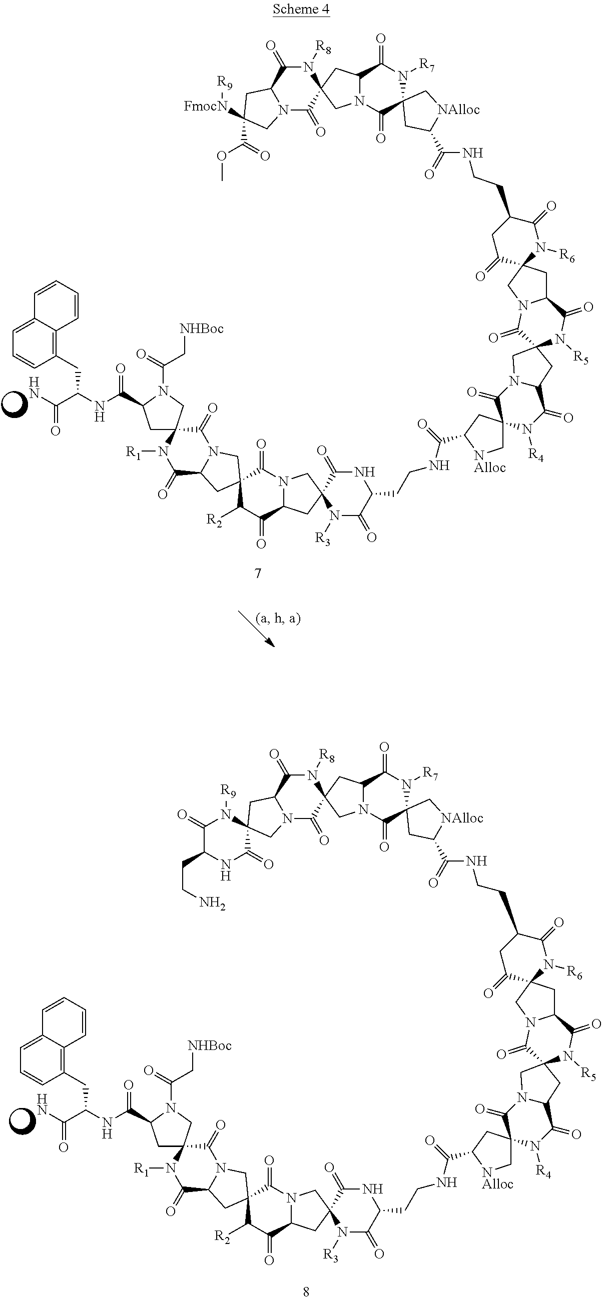

The resin bound bis-peptide three-mer was further elaborated to incorporate a bromoacetate group on the leading end amine to form 9. Bromoacetate was introduced according to procedure (M) using bromoacetic anhydride (23.4 mg, 90 μmoles), NMP (450 μL), and DIPEA (31.4 μL, 180 μmoles). The linear assembly of bis-peptides was liberated from the resin according to proceure (P) using 95% TFA/2.5% TIPS/2.5% Water (1.8 mL) to yield 9 as shown in Scheme 5.

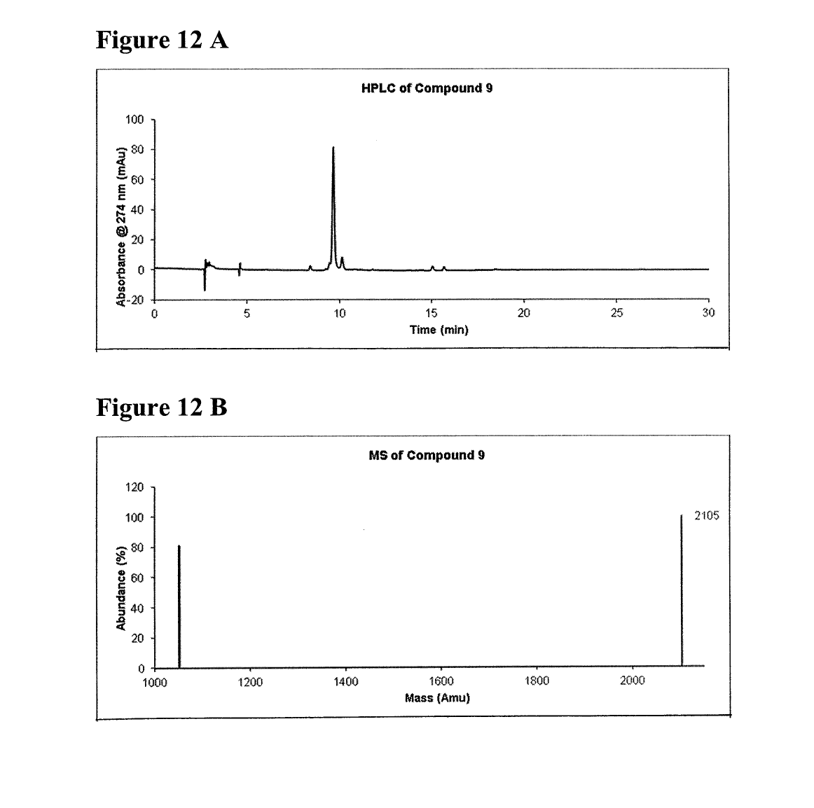

The reverse phase C18 HPLC-MS characterization of compound 9 where all the R₁ through R₉ groups are hydrogen is provided in FIG. 12.

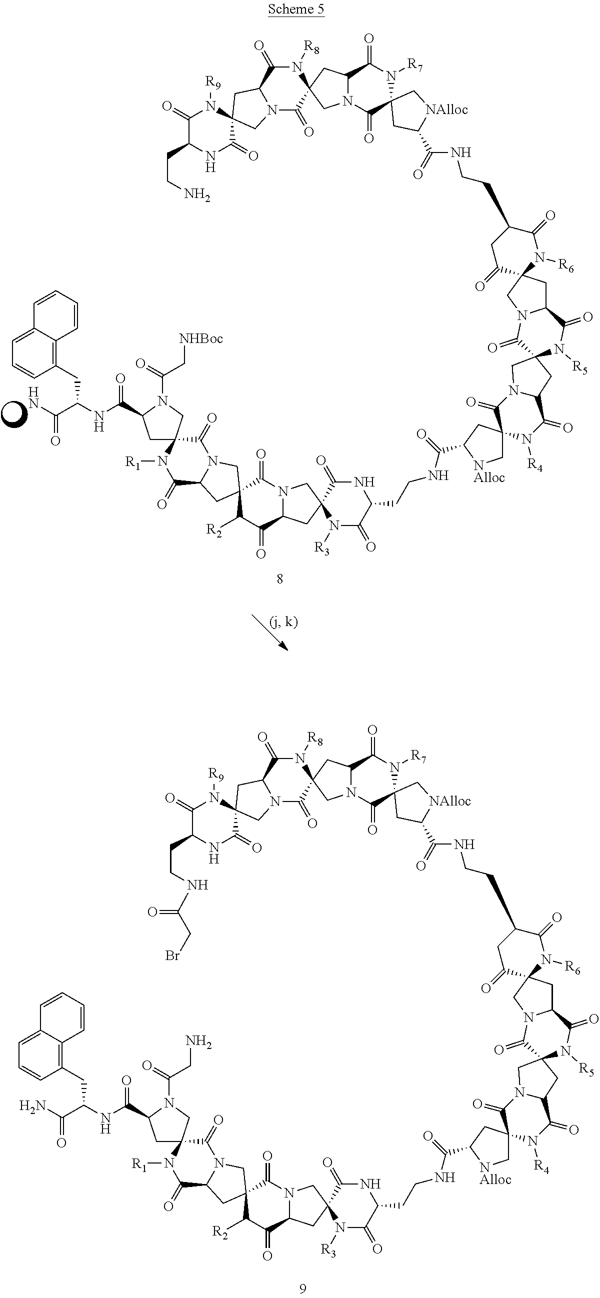

### Example 4

### Circular Macrocylces of Functionalized Bis-Peptides

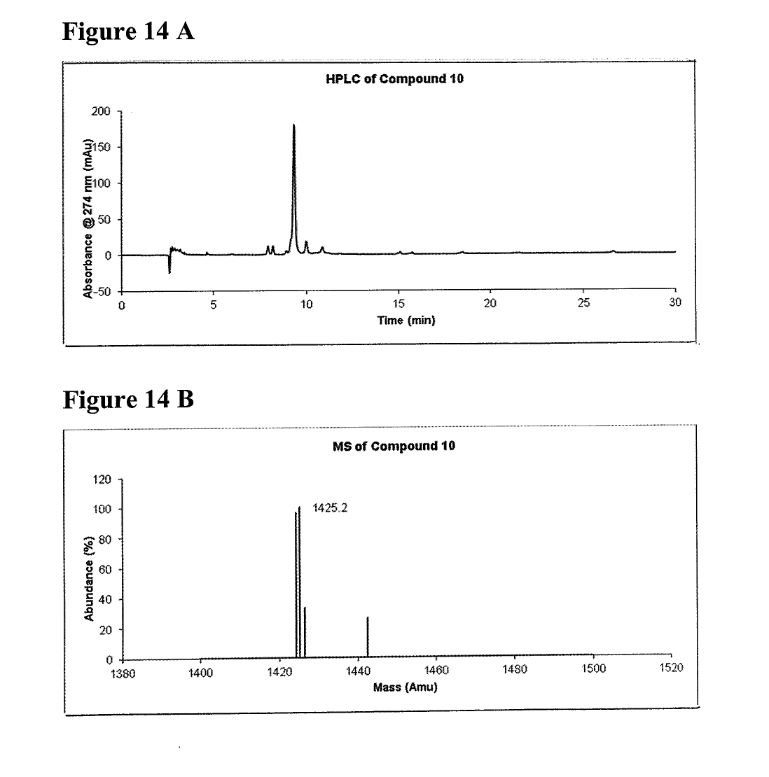

As an example of the synthesis of a functionalized macrocycle of bis-peptides, compound 6 (where R₁ to R₆ are hydrogen) was diluted slowly into a basic solution and the free primary amine of the glycine attacked the bromoacetate group on the leading end to create the two-bis-peptide containing macrocycle 10. Compound 6 (3.0 mg, 2 μmoles) was cross-linked according to general procedure [R] or [S] using DMF (44 mL) and 1.5% TEA in DMF (88 mL/μmole) or 50% MeCN in water (44 mL) and 0.05M pH7 Phosphate Buffer (88 mL) to yield compound 10 shown in Scheme 6. The reverse phase C18 HPLC-MS characterization of compound 10 is provided in FIG. 14.

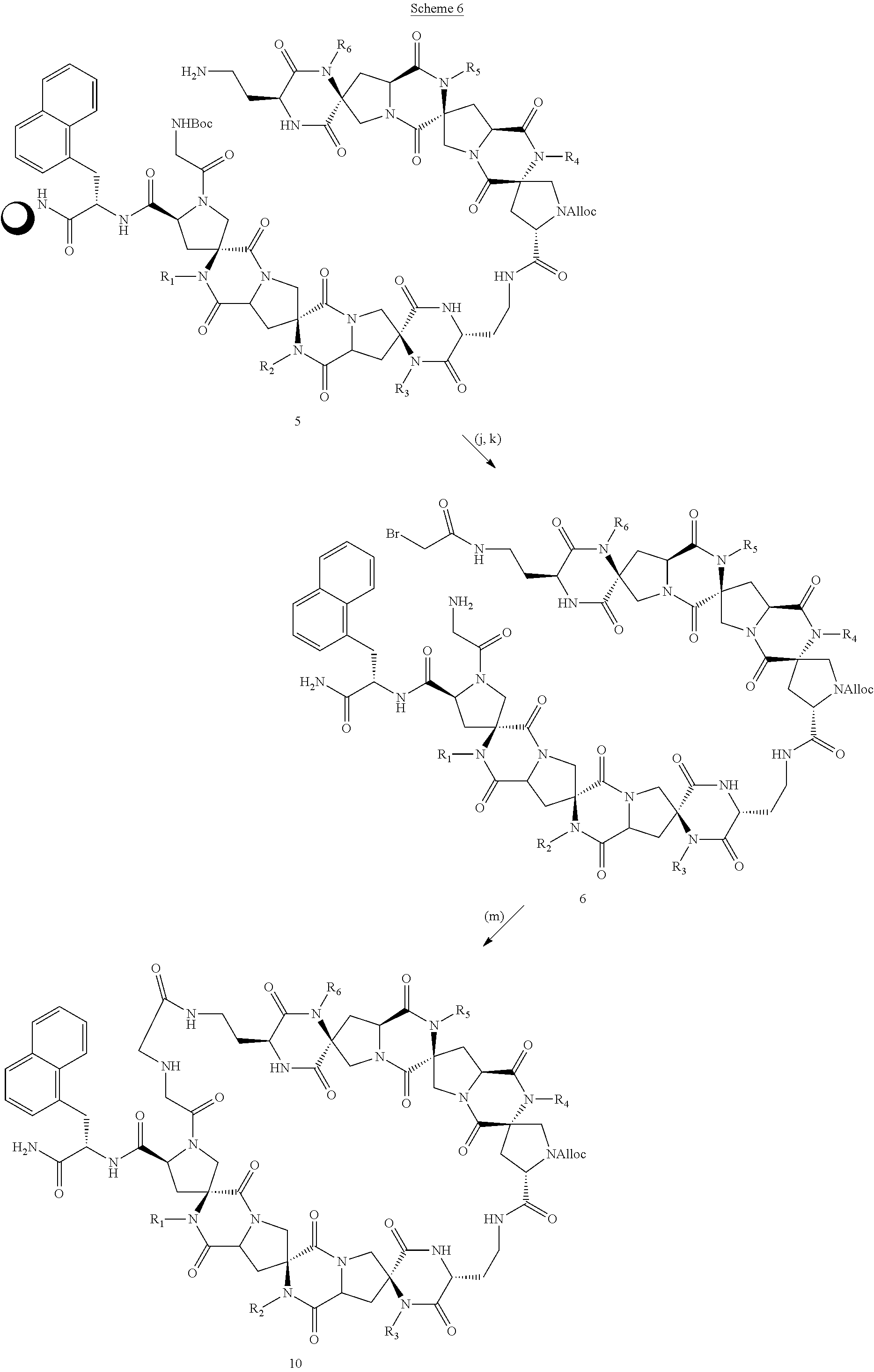

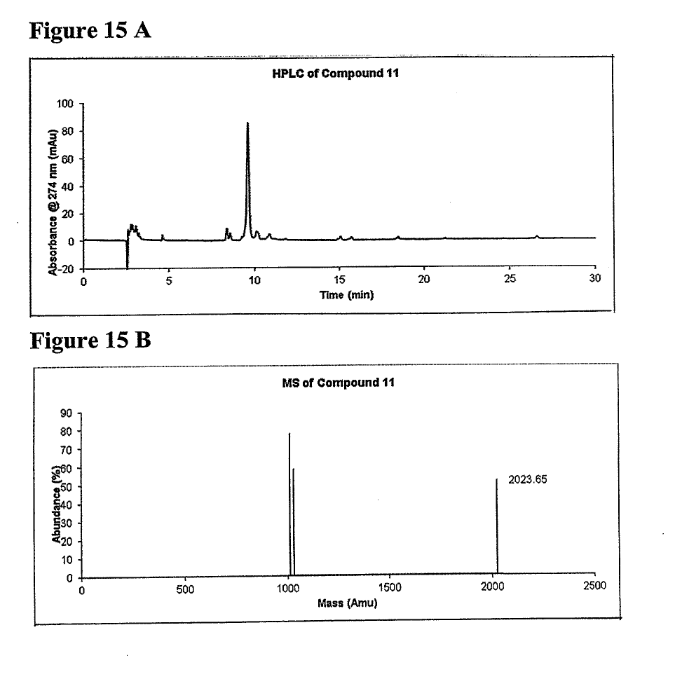

As an example of the synthesis of a larger functionalized macrocycle that has a large internal volume compound 9 was diluted slowly into a basic solution to create the three-bis-peptide containing macrocycle 11 of Scheme 7. Compound 9 (4.2 mg, 2 μmoles) was cross-linked according to general procedure [R] or [S] using DMF (44 mL) and 1.5% TEA in DMF (88 mL/μmole) or 50% MeCN in water (44 mL) and 0.05M pH7 Phosphate Buffer (88 mL) to yield compound 11. The reverse-phase HPLC-MS characterization of compound 11 is shown in FIG. 15.

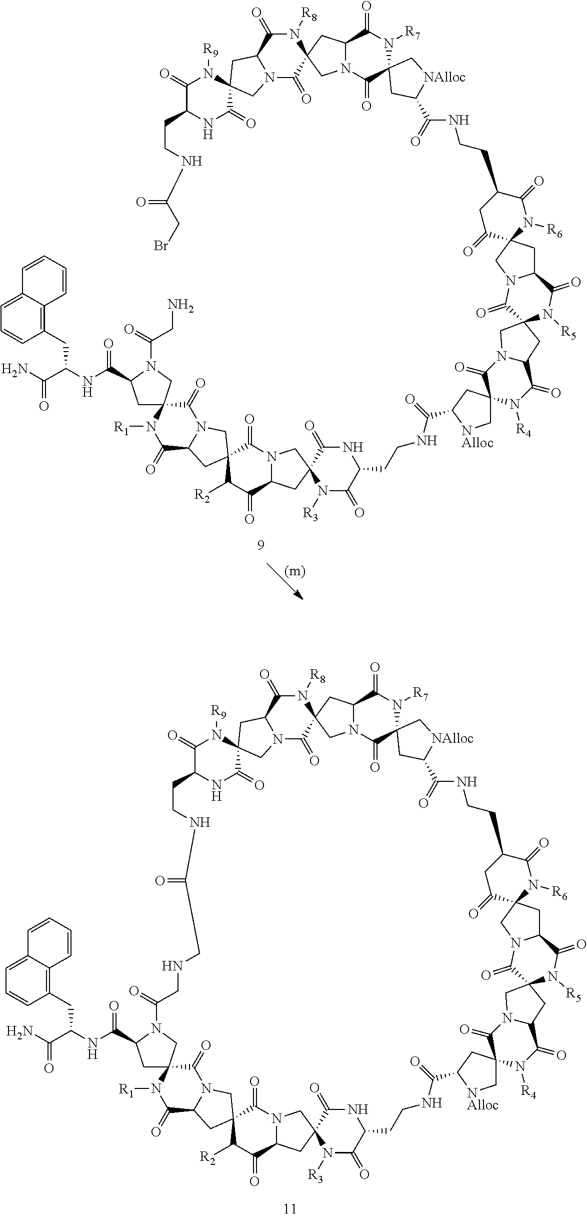

### Example 5

### Complex Three-Dimensional Network

To demonstrate the synthesis of a complex three-dimensional network of functionalized bis-peptides that displays a small molecule sized cavity, compound 7 was coupled to an N-Boc-N-Fmoc diaminobutanoic acid through a diketopiperazine ring as shown in Scheme 8. The two Alloc groups were then removed from the oligomer and an Fmoc-glycine was coupled to the two secondary amines that were revealed.

More specifically, Fmoc-L-Dab(Boc)-OH (39.6 mg, 90 μmoles) was coupled to 7 after Fmoc deprotection using procedure (J) according to general procedure (F) using HATU (34.2 mg, 90 μmoles), NMP (450 μL) and DIPEA (31.4 μL, 180 μmoles). The terminal Fmoc group was removed according to general procedure (J) using 20% piperidine in DMF (900 mL). Exposure to the deprotection solution was extended to 2 hours to enable complete diketopiperazine closure.

The Alloc group was removed according to general procedure (K) using borane:dimethylamine complex (10.6 mg, 180 μmoles) in DCM (450 μL) and tetrakis(triphenylphosiphine)palladium(0) (10.4 mg, 9 μmoles) in DCM (450 μL). Fmoc-Gly-OH (53.5 mg, 180 μmoles) was coupled according to general procedure (F) using HATU (68.4 mg, 180 μmoles), NMP (900 μL) and DIPEA (62.8 μL, 360 μmoles).

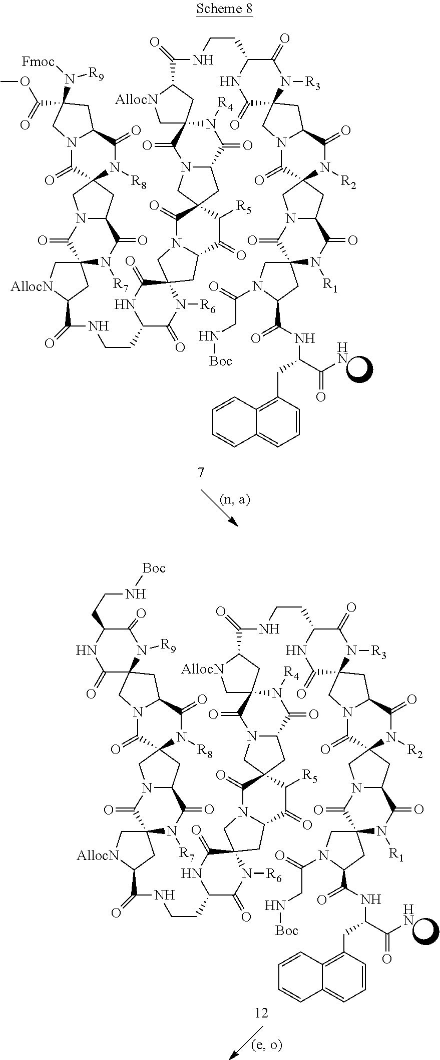

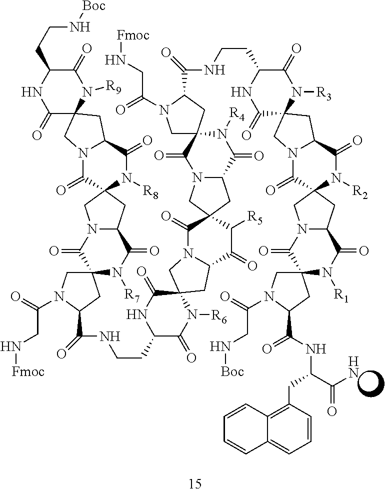

According to Scheme 9, the two Fmoc groups in 13 were removed and a bromoacetate group was coupled to each of the glycine amines that were revealed. Bromoacetates were introduced according to general procedure (M) using bromoacetic anhydride (46.8 mg, 180 μmoles), NMP (900 μL), and DIPEA (62.8 μL, 360 μmoles). The linear assembly of bis-peptides was liberated from the resin according to general procedure (P) using 95% TFA/2.5% TIPS/2.5% Water (1.8 mL) to yield 14.

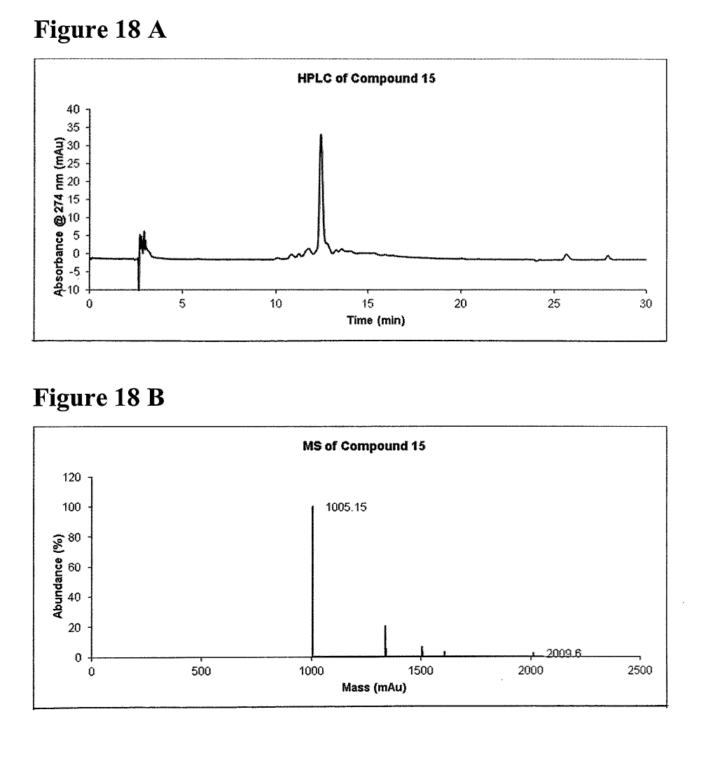

Compound 14 was then slowly added to a basic solution. Each of the amines reacted with only one bromoacetate group to form the double macrocycle 15, as illustrated in Scheme 9. Compound 14 (4.1 mg, 2 μmoles) was cross-linked according to general procedure [R] or [S] using DMF (44 mL) and 1.5% TEA in DMF (88 mL/μmole) or 50% MeCN in water (44 mL) and 0.05M pH7 Phosphate Buffer (88 mL) to form compound 15. The reverse phase C18 HPLC-MS characterization of compound 15 is provided in FIG. 18.

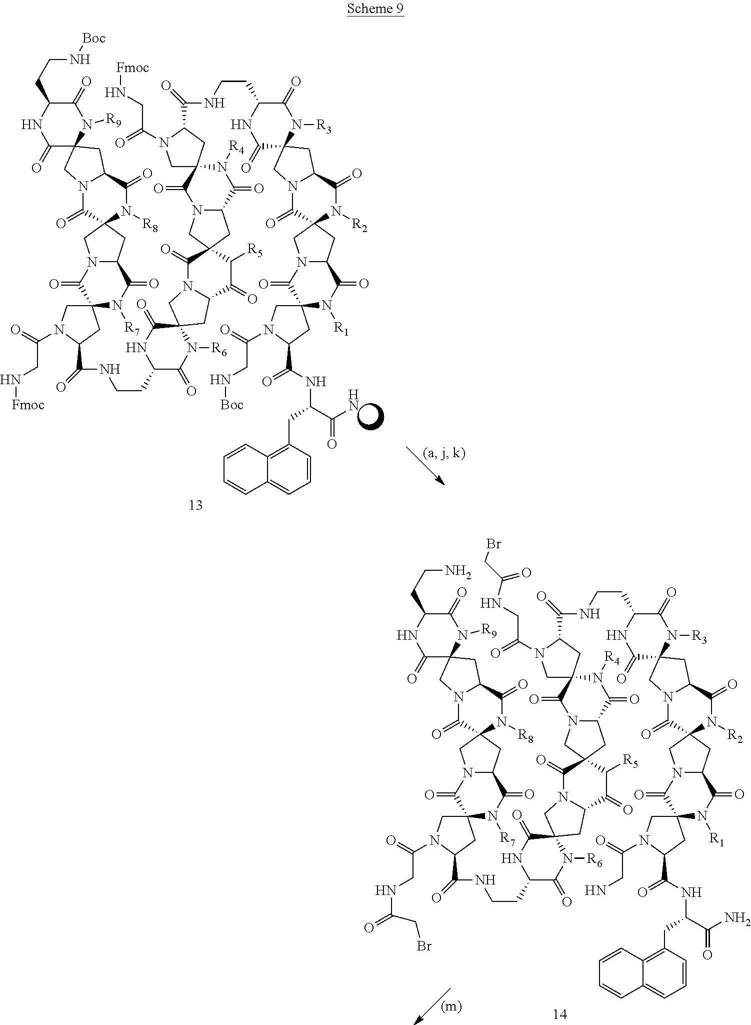

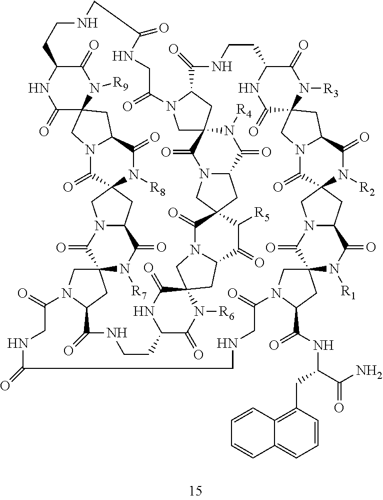

### Example 6

### Branched Bis-Peptide Nanostructures

In the synthesis of branched bis-peptide arrays three bis-peptides were coupled through an amide linkage to a polyamine. Tris(aminoethyl)amine was used to make a 3 oligomer branched structure, as illustrated in Scheme 10.

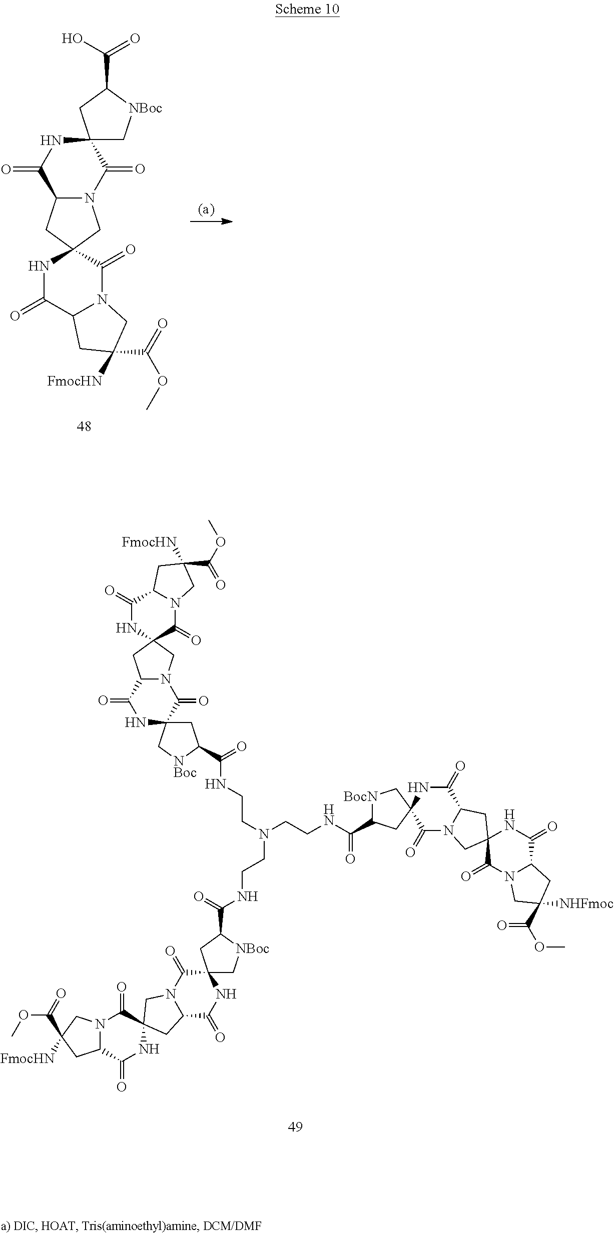

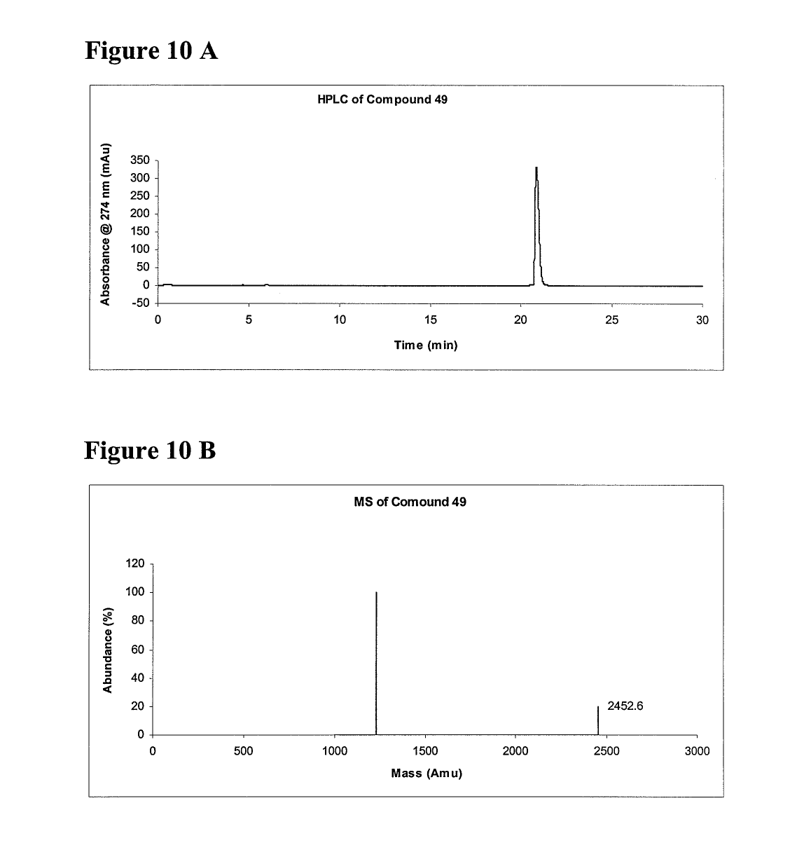

Bis-peptide 48 (69.4 mg, 90 μmoles) was activated according to procedure (E) using HOAT (73.5 mg, 540 μmoles), 1:2 DMF/DCM (1.6 mL), and DIC (14.1 μL, 90 μmoles). This reaction mixture was added to Tris(2-aminoethyl)amine (4.5 μL, 30 μmoles) in DMF (530 μL) to yield 49. The reverse phase C18 HPLC-MS characterization of compound 49 is shown in FIG. 10.

### Example 7

### Functionalized Bis-Peptide Macrocycle

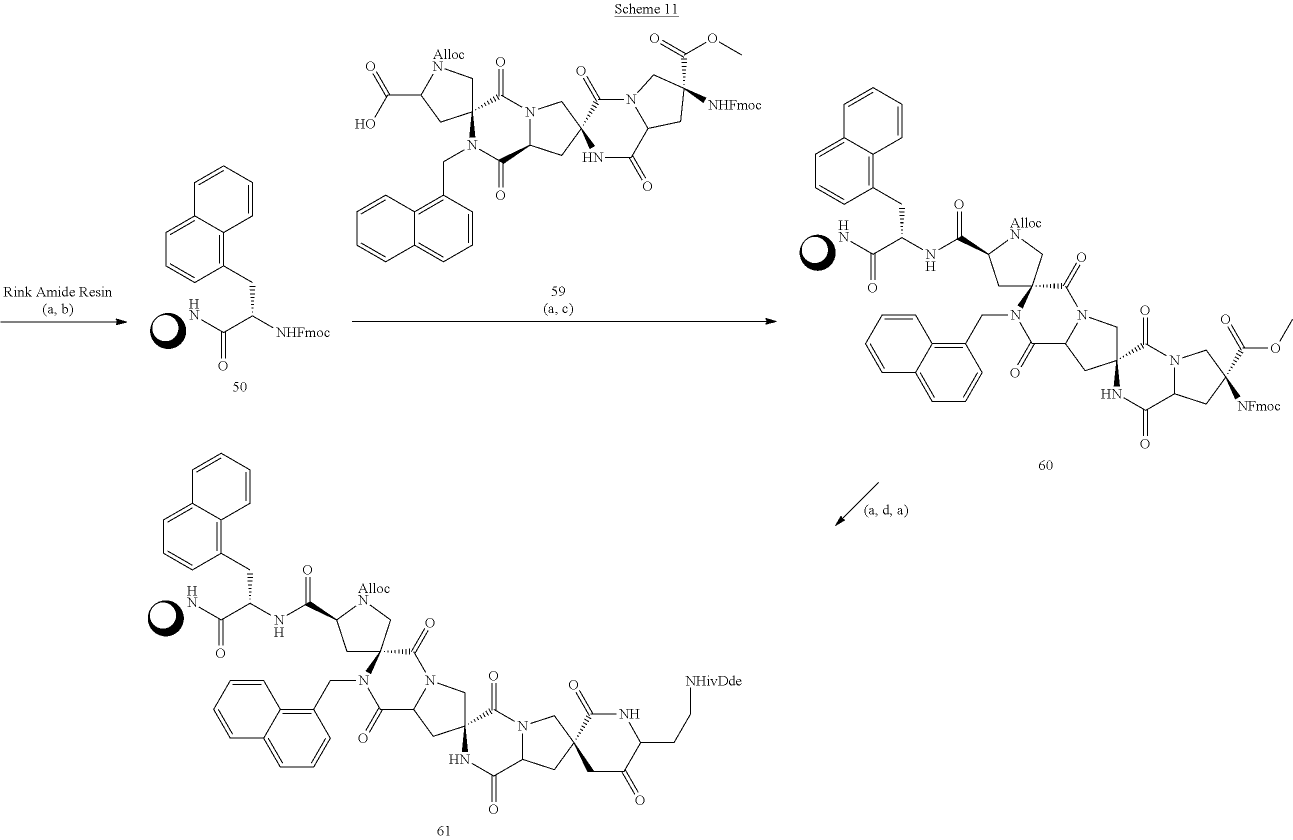

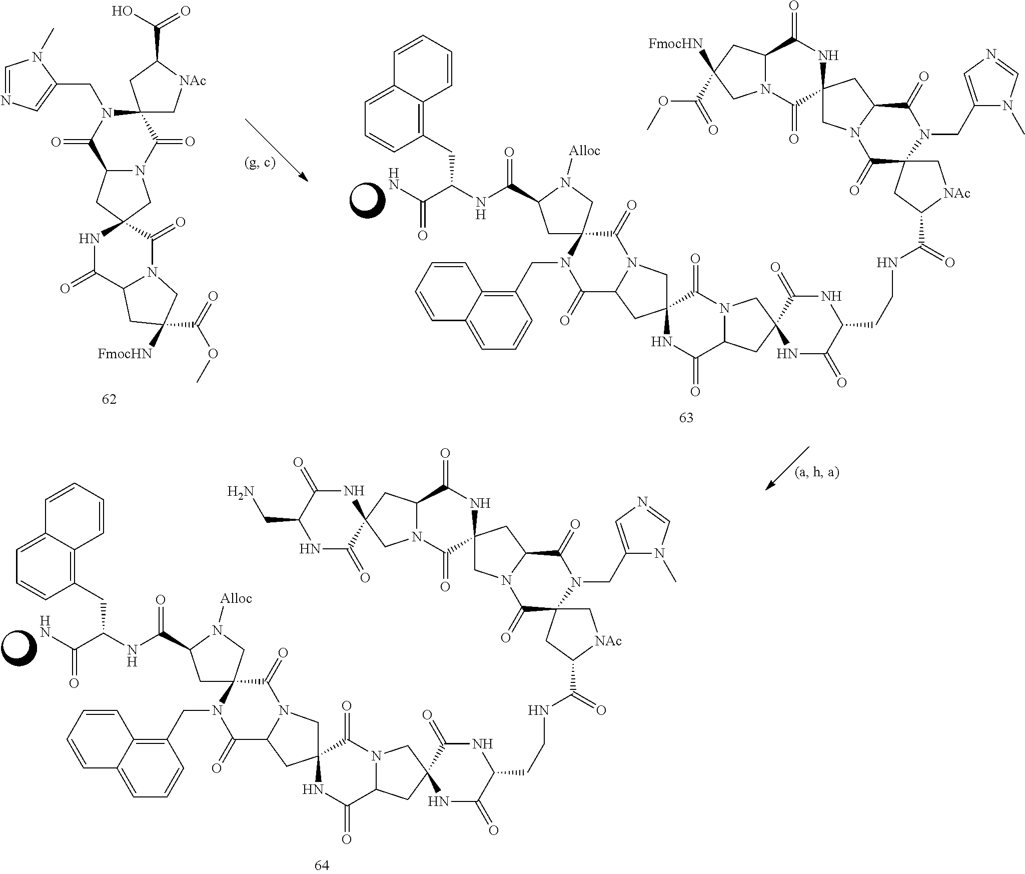

NovaPEG Rink Amide Resin (81.1 mg, 30 μmoles loading) was placed in an 8 mL solid phase reactor. (S)—N-Fmoc-1-Naphthylalanine-OH (39.4 mg, 90 μmoles) was attached according to general procedure (C) using HATU (34.2 mg, 90 μmoles), NMP (450 μL) and DIPEA (31.4 μL, 180 μmoles). The terminal Fmoc group was removed according to general procedure (J) using 20% piperidine in DMF (900 μL).

Compound 59 (82.0 mg, 90 μmoles) was coupled according to general procedure (G) using PyAOP (46.9 mg, 90 μmoles), NMP (450 μL), and DIPEA (31.4 μL, 180 μmoles). The terminal Fmoc group was removed according to general procedure (J) using 20% piperidine in DMF (900 μL).

Fmoc-D-Dab(IvDde)-OH (49.2 mg, 90 μmoles) was coupled according to general procedure (F) using HATU (34.2 mg, 90 μmoles), NMP (450 μL) and DIPEA (31.4 μL, 180 μmoles). The terminal Fmoc group was removed according to general procedure (J) using 20% piperidine in DMF (900 μL). Exposure to the deprotection solution was extended to 2 hours to enable complete diketopiperazine closure. The terminal IvDde group was removed according to general procedure (L) using 2% Hydrazine in DMF (900 μL).

Compound 62 (74.1 mg, 90 μmoles) was coupled according to general procedure (G) using PyAOP (46.9 mg, 90 μmoles), NMP (450 μL), and DIPEA (31.4 μL, 180 μmoles). The terminal Fmoc group was removed according to general procedure (J) using 20% piperidine in DMF (900 μL).

Fmoc-L-Dpr(Boc)-OH (38.4 mg, 90 μmoles) was coupled according to general procedure (F) using HATU (34.2 mg, 90 μmoles), NMP (450 μL) and DIPEA (31.4 μL, 180 μmoles). The terminal Fmoc group was removed according to general procedure (J) using 20% piperidine in DMF (900 μL). Exposure to the deprotection solution was extended to 2 hours to enable complete diketopiperazine closure.

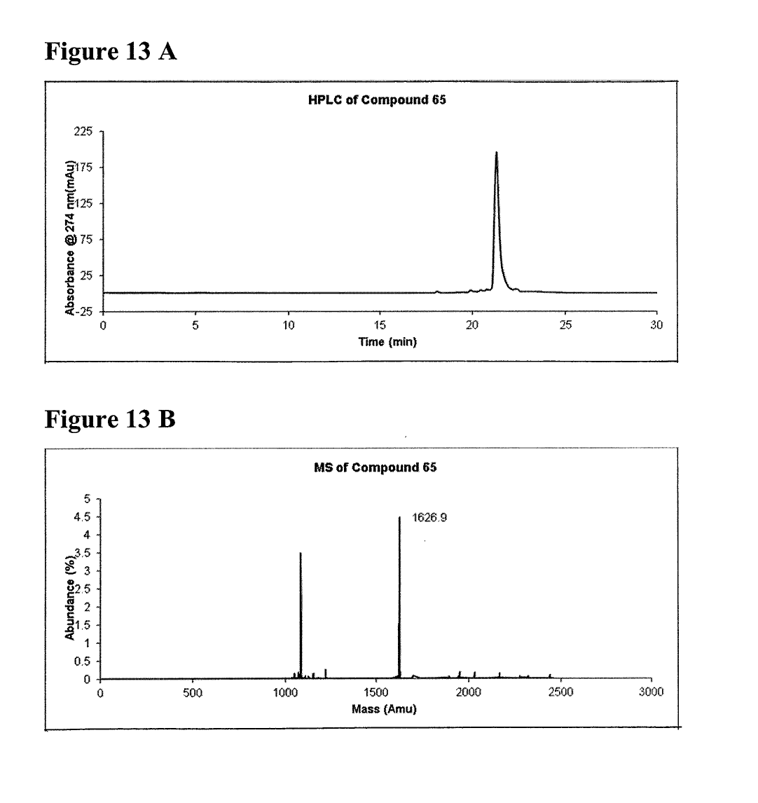

The Alloc group was removed according to general procedure (K) using borane:dimethylamine complex (10.6 mg, 180 μmoles) in DCM (450 μL) and tetrakis(triphenylphosiphine)palladium(0) (10.4 mg, 9 μmoles) in DCM (450 μL). Bromoacetate was introduced according to general procedure (M) using bromoacetic anhydride (23.4 mg, 90 μmoles), NMP (450 μL), and DIPEA (31.4 μL, 180 μmoles). The linear assembly of bis-peptides was liberated from the resin according to general procedure (P) using 95% TFA/2.5% TIPS/2.5% Water (1.8 mL) to yield 65 as shown in Scheme 12. The reverse phase C18 HPLC-MS characterization of compound 65 is shown in FIG. 13.

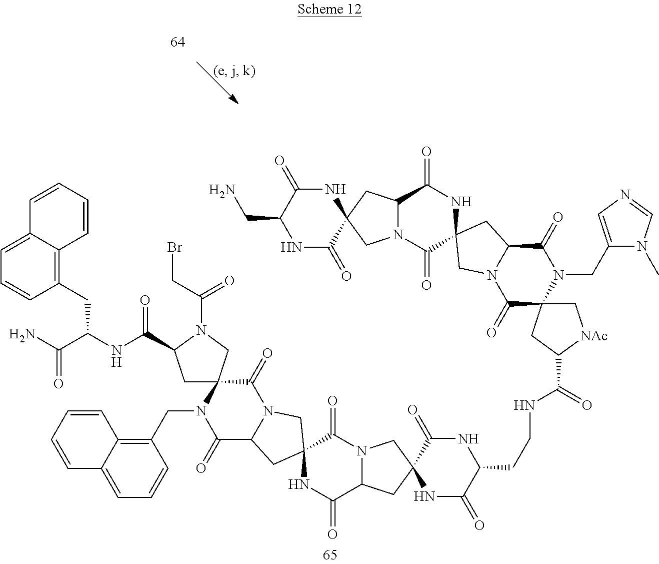

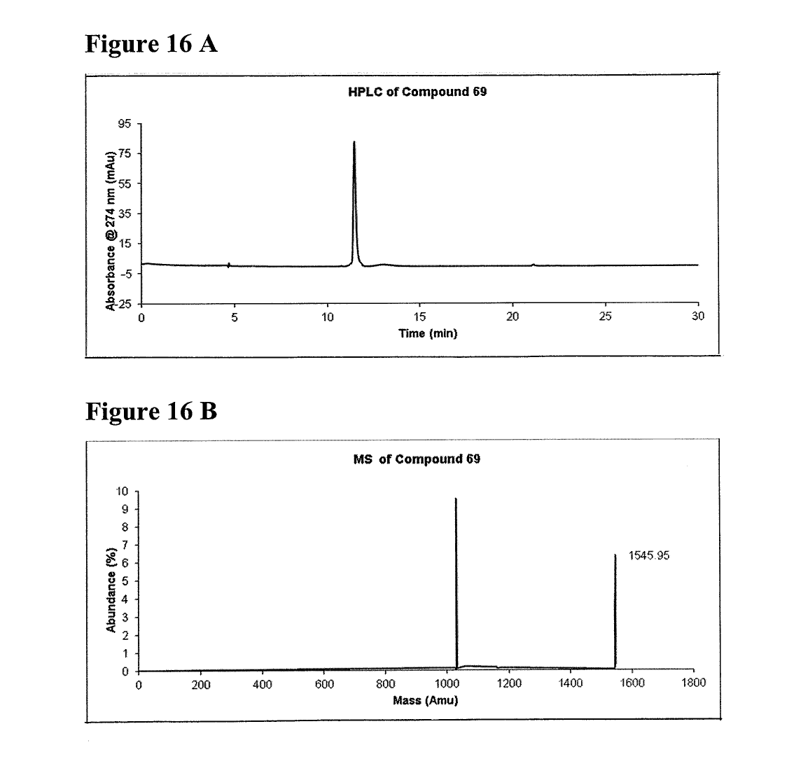

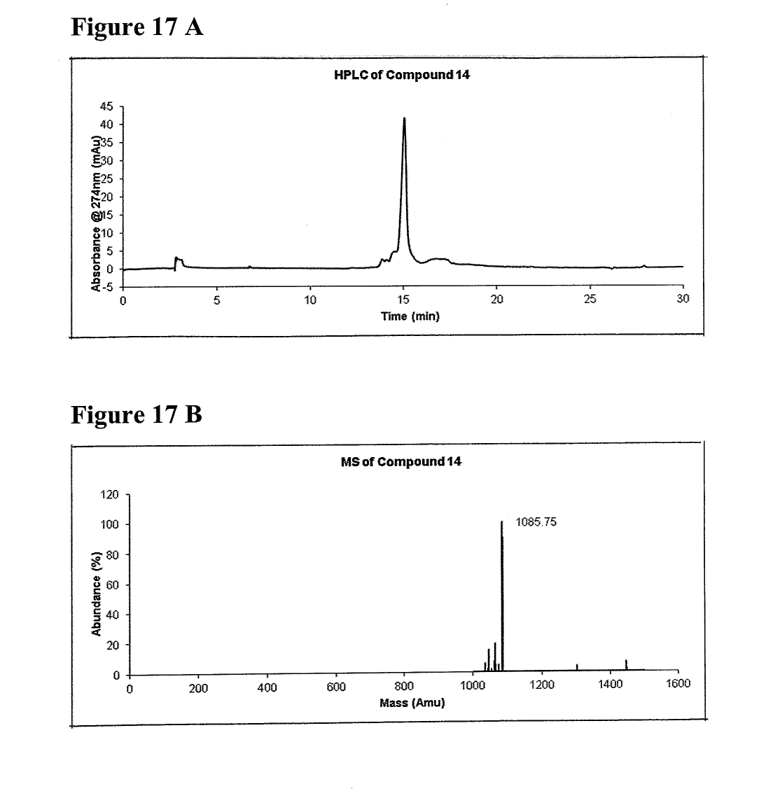

Compound 68 (3.3 mg, 2 mmoles) was cross-linked according to general procedure [R] or [S] using DMF (44 mL) and 1.5% TEA in DMF (88 mL/μmole) or 50% MeCN in water (44 mL) and 0.05M pH7 Phosphate Buffer (88 mL), as shown in Scheme 13. The reverse phase C18 HPLC-MS characterization of compound 69 is shown in FIG. 16.

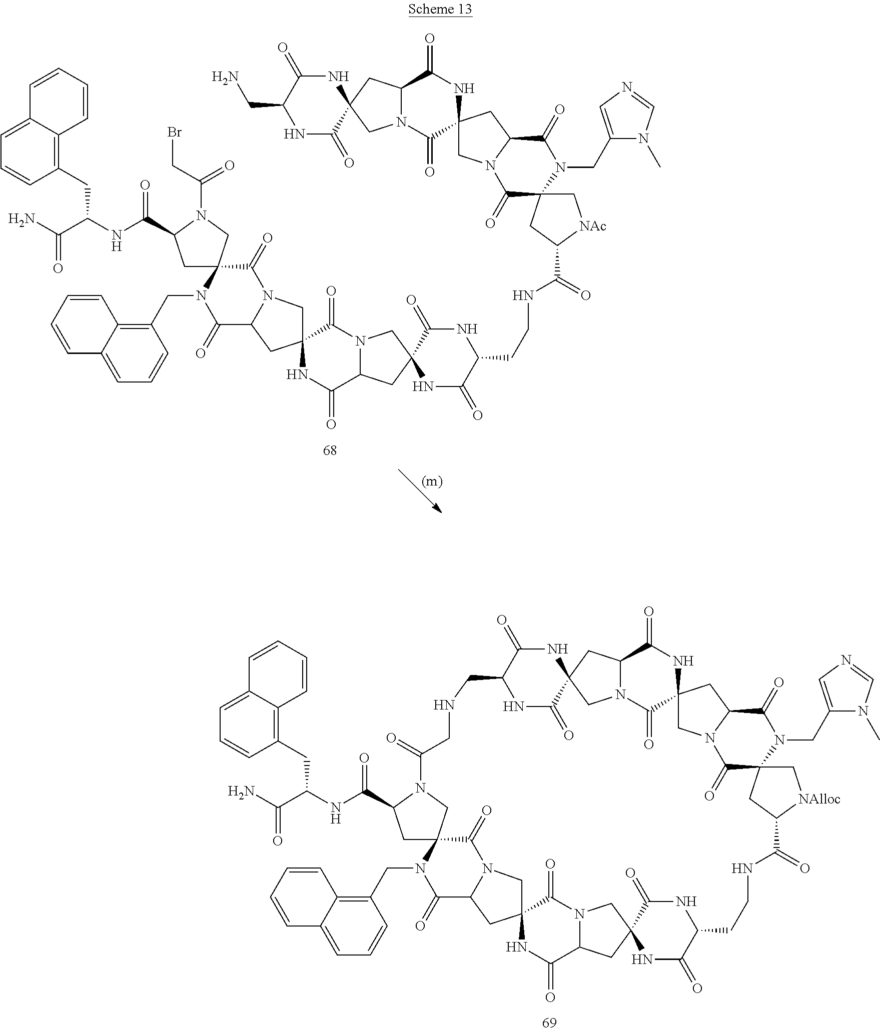

What is claimed is:

1. A macromolecule comprising two or more functionalized bis-peptides connected by one or more linkers.

2. The macromolecule of claim 1 wherein said functionalized bis-peptides are covalently attached to one or more functionalized bis-peptides to form linear strings of functionalized bis-peptides, macrocyles of functionalized bis-peptides, three-dimensional networks of functionalized bis-peptides, and combinations of any of these.

3. The macromolecule of claim 1 or 2 wherein said functionalized bis-peptides are connected by said one or more linkers to form a linear array.

4. The macromolecule of claim 1 or 2 wherein said functionalized bis-peptides are connected by said one or more linkers to form a branched array.

5. The macromolecule of claim 1 or 2 wherein said functionalized bis-peptides are connected by said one or more linkers to form a macrocycle.

6. The macromolecule of claim 1 or 2 wherein said functionalized bis-peptides are connected by said one or more linkers to form a three-dimensional network.

7. The macromolecule of claim 6 wherein said three-dimensional network comprises pre-organized pockets.

8. The macromolecule of claim 6 wherein said three-dimensional network comprises dendrimers.

9. The macromolecule of claim 1 or 2 wherein said functionalized bis-peptides are functionalized with reactive groups comprising amines, carboxylic acids, azides, alkynes, thiols, olefins, dienes, dienophiles, aldehydes, hydrazines, phosphines, maleimides, haloacetates, halobenzyl groups, alcohols, aziridines, epoxides, aryl halides or vinyl halides.

10. The macromolecule of claim 1 or 2 wherein one or more of said linkers are flexible.

11. The macromolecule of claim 1 or 2 wherein one or more of said linkers are branched.

12. The macromolecule of claim 1 or 2 wherein said one or more linkers comprise amides, esters, cis or trans alkenes, amino acids, amines, ethers, thioethers, acyls, allyls, propargyls, benzyls, hydrazines, triazoles, rings using Diels-Alder couplings, hydrazones, hydrazides, disulphide linkages, alkyl-chains, aromatic rings, olefins formed using metathesis, phosphonate ester linkages, silyl linkages, organometallic linkages and alkyl chain linkages and combinations thereof.

13. The macromolecule of claim 1 or 2 wherein said one or more linkers are formed by reaction of reactive groups located on the two or more bis-peptides.

14. The macromolecule of claim 13 wherein said reactive groups are located on opposite ends of said two or more bis-peptides.

15. The macromolecule of claim 13 wherein one said reactive group is located on an interior portion of said one or more bis-peptides and one said reactive group is located on an end of said two or more bis-peptides.

16. The macromolecule of claim 13 wherein two said reactive groups are located on an interior portion of said two or more bis-peptides.

17. The macromolecule of claim 13 wherein two said reactive groups are located on the same end of said two or more bis-peptides.

18. A method of making a macromolecule comprising at least two functionalized bis-peptides joined to each other through a flexible linker comprising covalently attaching a functionalized bis-peptide to one or more functionalized bis-peptides to form linear strings of functionalized bis-peptides, macrocycles of functionalized bis-peptides, three-dimensional networks of functionalized bis-peptides, and combinations of any of these.

19. A method of making a macromolecule comprising two or more functionalized bis-peptides connected by one or more linkers comprising reacting two or more reactive groups on the one or more functionalized bis-peptides to form said macromolecule.

20. The method of claim 18 or 19 wherein one or more of said linkers are flexible.

21. The method of claim 18 or 19 wherein one or more of said linkers are branched.

22. The method of claim 18 or 19 wherein said reactive groups are on opposite ends of said two or more bis-peptides.

23. The method of claim 18 or 19 wherein one said reactive group is on an interior portion of said one or more bis-peptides and one said reactive group is on an end of said two or more bis-peptides.

24. The method of claim 18 or 19 wherein two said reactive groups are on an interior portion of said two or more bis-peptides.

25. The method of claim 18 or 19 wherein two said reactive groups are on the same end of said two or more bis-peptides.

26. A macromolecule comprising two or more bis-peptides connected by one or more linkers.

27. The macromolecule of claim 26 wherein said bis-peptides comprise non-functionalized bis-peptides, functionalized bis-peptides or a combination of both functionalized and non-functionalized bis-peptides.

28. The macromolecule of claim 27 wherein said bis-peptides are covalently attached to one or more bis-peptides to form linear strings of bis-peptides, macrocyles of bis-peptides, three-dimensional networks of bis-peptides, and combinations of any of these.

29. The macromolecule of claim 27 wherein said bis-peptides are connected by said one or more linkers to form a linear array.

30. The macromolecule of claim 27 wherein said bis-peptides are connected by said one or more linkers to form a branched array.

31. The macromolecule of claim 27 wherein said bis-peptides are connected by said one or more linkers to form a macrocycle.

32. The macromolecule of claim 27 wherein said bis-peptides are connected by said one or more linkers to form a three-dimensional network.

33. A method of making a macromolecule comprising two or more bis-peptides connected by one or more linkers comprising reacting two or more reactive groups on the one or more bis-peptides to form said macromolecule.

34. The macromolecule of claim 33 wherein said bis-peptides comprise non-functionalized bis-peptides, functionalized bis-peptides or a combination of both functionalized and non-functionalized bis-peptides.

35. The method of claim 34 wherein one or more of said linkers are flexible.

36. The method of claim 34 wherein one or more of said linkers are branched.
